(19) United States
(12) Patent Application Publication
(10) Pub. No.: US 2011/0212958 A1
(43) Pub. Date: Sep. 1, 2011

(54) FATTY ACID RALOXIFENE DERIVATIVES AND THEIR USES

(75) Inventors: Jill C. Milne, Brookline, MA (US); Michael R. Jirousek, Cambridge, MA (US); Jean E. Bemis, Arlington, MA (US); Chi B. Vu, Arlington, MA (US)

(73) Assignee: CATABASIS PHARMACEUTICALS, INC., Cambridge, MA (US)

(21) Appl. No.: 13/035,627

(22) Filed: Feb. 25, 2011

Related U.S. Application Data
(60) Provisional application No. 61/308,763, filed on Feb. 26, 2010.
(60) Provisional application No. 61/310,959, filed on Mar. 5, 2010.

(51) Int. Cl. A61K 31/5377; C07D 413/12; C07D 409/12; A61K 31/4535; A61P 19/10; A61P 3/06; A61P 35/00; A61P 15/00; A61P 9/00

(52) U.S. Cl. 514/233.5; 544/146; 546/202; 514/324

(57) ABSTRACT

The invention relates to fatty acid raloxifene derivatives; compositions comprising an effective amount of a fatty acid raloxifene derivative; and methods for treating osteoporosis or preventing invasive breast cancer in postmenopausal women comprising the administration of an effective amount of a fatty acid raloxifene derivative.

### PRIORITY

The present application claims the benefit of U.S. Provisional Application No. 61/308,763 filed Feb. 26, 2010, and U.S. Provisional Application No. 61/310,959 filed Mar. 5, 2010. The entire disclosures of those applications are relied on for all purposes and are incorporated into this application by reference.

### FIELD OF THE INVENTION

The invention relates to fatty acid raloxifene derivatives; compositions comprising an effective amount of a fatty acid raloxifene derivative; and methods for the treatment of osteoporosis, endometriosis, uterine fibrosis, metabolic dyslipidemia, coronary heart disease and for the prevention of invasive breast cancer in postmenopausal women comprising the administration of an effective amount of a fatty acid raloxifene derivative. All patents, patent applications, and publications cited herein are hereby incorporated by reference in their entireties.

### BACKGROUND OF THE INVENTION

Oily cold water fish, such as salmon, trout, herring, and tuna are the source of dietary marine omega-3 fatty acids, with eicosapentaenoic acid (EPA) and docosahexaenoic acid (DHA) being the key marine derived omega-3 fatty acids. Omega-3 fatty acids have previously been shown to improve insulin sensitivity and glucose tolerance in normoglycemic men and in obese individuals. Omega-3 fatty acids have also been shown to improve insulin resistance in obese and non-obese patients with an inflammatory phenotype. Lipid, glucose, and insulin metabolism have been shown to improve in overweight hypertensive subjects through treatment with omega-3 fatty acids. Omega-3 fatty acids (EPA/DHA) have also been shown to decrease triglycerides and to reduce the risk for sudden death caused by cardiac arrhythmias in addition to improve mortality in patients at risk of a cardiovascular event. Omega-3 fatty acids have also been taken as part of the dietary supplement portion of therapy used to treat dyslipidemia. Last, but not least, omega-3 fatty acids have been known to have a number of anti-inflammatory properties. For instance, a higher intake of omega-3 fatty acids has been shown to lower levels of circulating TNFα and IL-6, two of the cytokines that are markedly increased during inflammation processes (Chapkin et al, Prostaglandins, Leukot Essent Fatty Acids 2009, 81, p. 187-191; Duda et al, Cardiovasc Res 2009, 84, p. 33-41). In addition, a higher intake of omega-3 fatty acids has also been shown to increase levels of the well-characterized anti-inflammatory cytokine IL-10 (Bradley et al, Obesity (Silver Spring) 2008, 16, p. 938-944). More recently, there is additional evidence that omega-3 fatty acids could play a significant role in oncology (For reviews see: Gleissman, H. et al Experimental Cell Research 2010, 316, p. 1365-73; Bougnoux, P. et al Progress in Lipid Research 2010, 49, p. 76-86; Spencer, L. et al, Eur. J. Cancer 2009, 45, p. 2077-86; Serini, S. et al Apoptosis 2009, 14, p. 135-152; Browever, I. A. Prostaglandins, Leukotrienes and Essential Fatty Acids 2008, 79, p. 97-99). In a study using the xenograft model in nude mice, treatment with omega-3 fatty acids, such as DHA and EPA, resulted in breast tumor regression. Here, treatment with DHA/EPA appeared to increase the level of PTEN protein and attenuate the PI 3 kinase and Akt kinase activity as well as the expression of the anti-apoptotic proteins Bcl-2 and Bcl-XL in the breast tumors (Ghosh-Choudhury, T. et al. Breast Cancer Res. Treat. 2009, 118 (1), 213-228). Additional evidence supporting the use of omega-3 fatty acids in oncology also appeared in a recent study by Lim et al. showing that DHA/EPA could inhibit hepatocellular carcinoma cell growth, presumably by blocking β-catenin and cyclooxygenase-2 (Lim, K. et al. Mol. Cancer. Ther. 2009, 8 (11), 3046-3055).

Both DHA and EPA are characterized as long chain fatty acids (aliphatic portion between 12-22 carbons). Medium chain fatty acids are characterized as those having the aliphatic portion between 6-12 carbons. Lipoic acid is a medium chain fatty acid found naturally in the body. It plays many important roles such as free radical scavenger, chelator to heavy metals and signal transduction mediator in various inflammatory and metabolic pathways, including the NF-κB pathway (Shay, K. P. et al. Biochim. Biophys. Acta 2009, 1790, 1149-1160). Lipoic acid has been found to be useful in a number of chronic diseases that are associated with oxidative stress (for a review see Smith, A. R. et al Curr. Med. Chem. 2004, 11, p. 1135-46). Lipoic acid has now been evaluated in the clinic for the treatment of diabetes (Morcos, M. et al Diabetes Res. Clin. Pract. 2001, 52, p. 175-183) and diabetic neuropathy (Mijnhout, G. S. et al Neth. J. Med. 2010, 110, p. 158-162). Lipoic acid has also been found to be potentially useful in treating cardiovascular diseases (Ghibu, S. et al, J. Cardiovasc. Pharmacol. 2009, 54, p. 391-8), Alzheimer's disease (Maczurek, A. et al, Adv. Drug Deliv. Rev. 2008, 60, p. 1463-70) and multiple sclerosis (Yadav, V. Multiple Sclerosis 2005, 11, p. 159-65; Salinthone, S. et al, Endocr. Metab. Immune Disord. Drug Targets 2008, 8, p. 132-42).

Raloxifene is a selective estrogen receptor modulator (SERM) that has been approved by the FDA for the treatment of osteoporosis and to lower the risk of invasive breast cancer in postmenopausal women. Raloxifene was specifically developed in order to maintain the beneficial estrogenic activity on bone and lipids and to exert anti-estrogenic activity on endometrial and breast tissue. Raloxifene works by inducing conformational changes in the estrogen receptor, which in turn, enables the expression of certain estrogen-regulated genes in different tissues. For instance, the agonist properties of raloxifene on bone tissues were recently attributed to the activation of the human transforming growth factor-b3 gene, which is generally regarded to be essential in bone remodeling. In clinical trials, women receiving 30, 60 and 150 mg of raloxifene a day for 24 months showed significant increases in bone mineral density in the lumbar spine, total hip, femoral neck and total body, when compared with women who received the placebo (Delmas, P. D. et al. New Engl. J. Med. 1997, 337 (23), 1641-1647). Studies have also shown that a number of pro-inflammatory cytokines such as IL-1, TNFα and IL-6 are elevated during accelerated bone loss at menopause; and raloxifene could potentially reduce this inflammation state by its ability to block the PI3 kinase/Akt-NFκB signaling cascade (Lee et al. Mol. Cells. 2008, 26, 48-52). In the Study of Tamoxifen and raloxifene (STAR) clinical study involving 19,747 high risk postmenopausal women, raloxifene has been further shown to work as well as Tamoxifen in reducing the risk of invasive breast cancer with fewer side effects.

The ability to provide the effects of fatty acids and raloxifene in a synergistic way would provide benefits in treating osteoporosis and for lowering the risk of invasive breast cancer in postmenopausal women.

### SUMMARY OF THE INVENTION

The invention is based in part on the discovery of fatty acid raloxifene derivatives and their demonstrated effects in achieving improved treatment that cannot be achieved by administering raloxifene or fatty acids alone or in combination. These novel compounds are useful in the treatment of osteoporosis, endometriosis, uterine fibrosis, metabolic dyslipidemia, coronary heart disease and for the prevention of invasive breast cancer in postmenopausal women.

In another aspect, compounds of the Formula I are described:

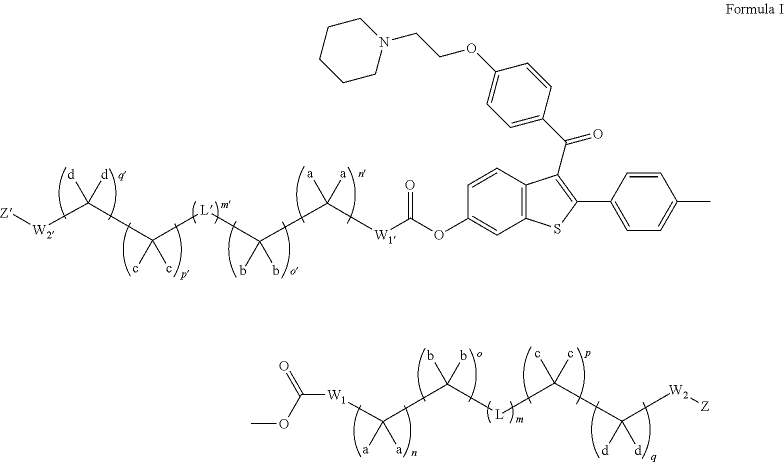

and pharmaceutically acceptable salts, hydrates, solvates, prodrugs, enantiomers and stereoisomers thereof;

wherein

W₁, W_{1′}, W₂, and W_{2′} are each independently null, O, S, NH, NR, or, W₁ and W₂ or W_{1′} and W_{2′} can be taken together can form an imidazolidine or piperazine group,

each a, b, c, and d is independently —H, , —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;

each n, n′, o, o′, p, p′, q, and q′ is independently 0, 1 or 2;

each L and L′ is independently null, —O—, —S—, —S(O)—, —S(O)₂—, —S—S—, —(C₁-C₆alkyl)-, —(C₃-C₆cycloalkyl)-, a heterocycle, a heteroaryl,

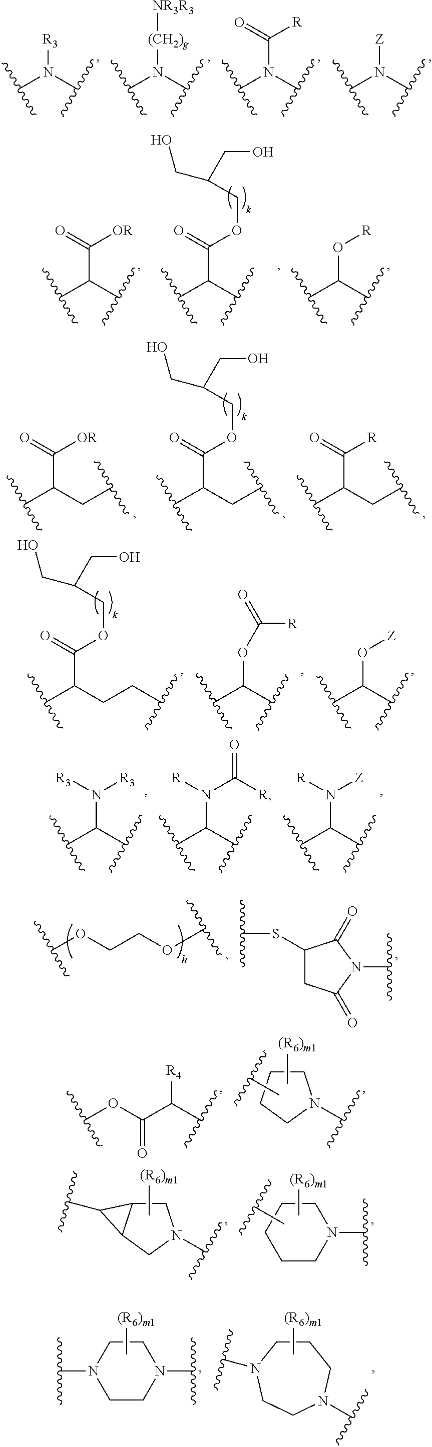

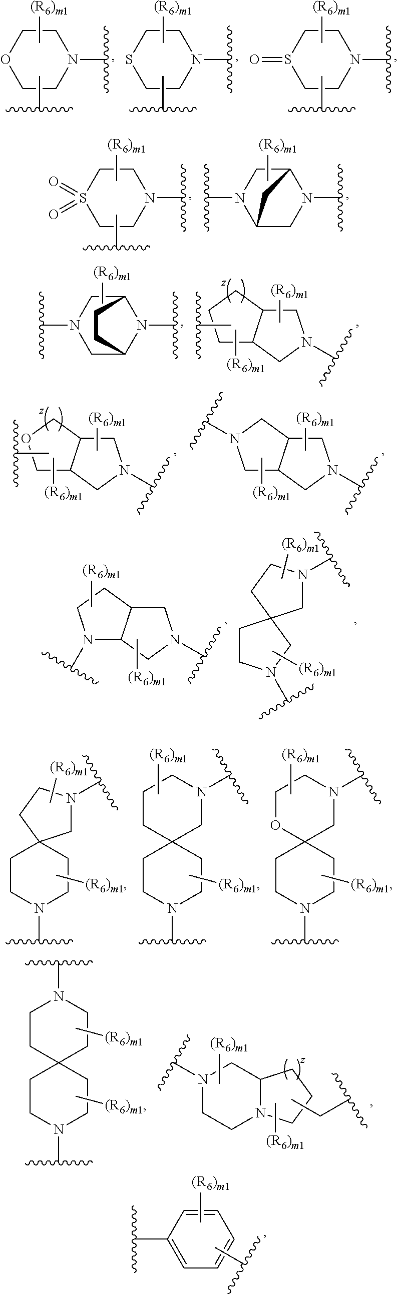

wherein the representation of L is not limited directionally left to right as is depicted, rather either the left side or the right side of L can be bound to the W₁ side of the compound of Formula I;

R₆ is independently —H, , —C₁-C₄ alkyl, gen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m and m′ is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

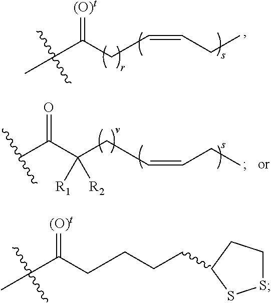

with the proviso that there is at least one

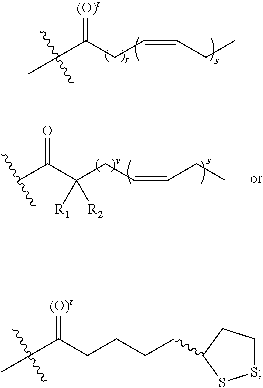

in the compound;

each r is independently 2, 3, or 7;

each s is independently 3, 5, or 6;

each t is independently 0 or 1;

each v is independently 1, 2, or 6;

each R₁ and R₂ are independently hydrogen, deuterium, —C₁-C₄ alkyl, gen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

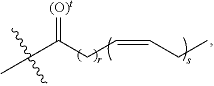

then t must be 0;
when m, n, o, p, and q are each 0, and W₁ and W₂ are each null, then Z must not be

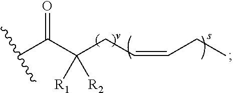

when m′, n′, o′, p′, and q′ are each 0, W_{1′} and W_{2′} are each null, and Z′ is

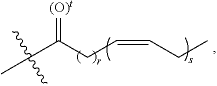

then t must be 0;
when m′, n′, o′, p′, and q′ are each 0, and W_{1′} and W_{2′} are each null, then Z′ must not be

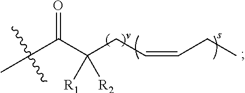

and
   when m, m′, n, n′, o, o′, p, p′, q, and q′ are each 0, W₁, W₂, W_{1′}, and W_{2′} are each null, each t is 1, Z and Z′ are each

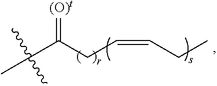

and each r is 7, then one s must be 5 or 6.

In another aspect, compounds of the Formula II are described:

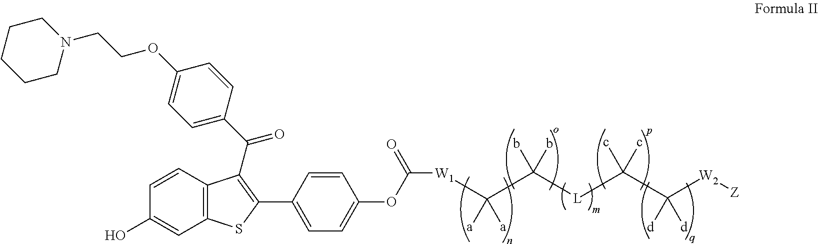

and pharmaceutically acceptable salts, hydrates, solvates, prodrugs, enantiomers and stereoisomers thereof;

wherein

W₁ and W₂ are each independently null, O, S, NH, NR, or W₁ and W₂ can be taken together can form an imidazolidine or piperazine group,

each a, b, c, and d is independently —H, , —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;

each n, o, p, and q is independently 0, 1 or 2;

L is independently null, —O—, —S—, —S(O)—, —S(O)₂—, —S—S—, —(C₁-C₆alkyl)-, —(C₃-C₆cycloalkyl)-, a heterocycle, a heteroaryl,

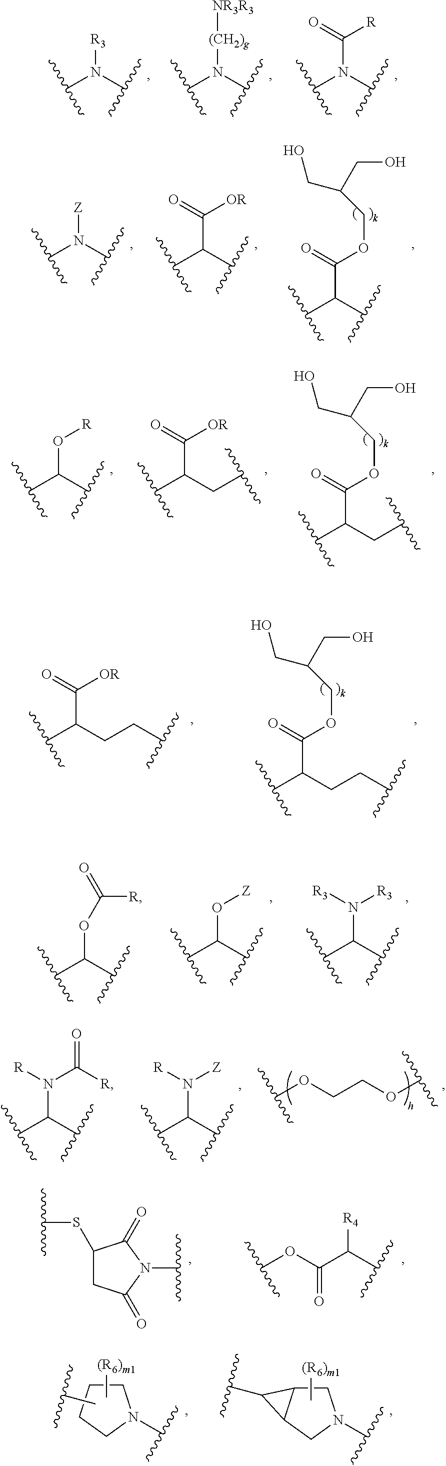

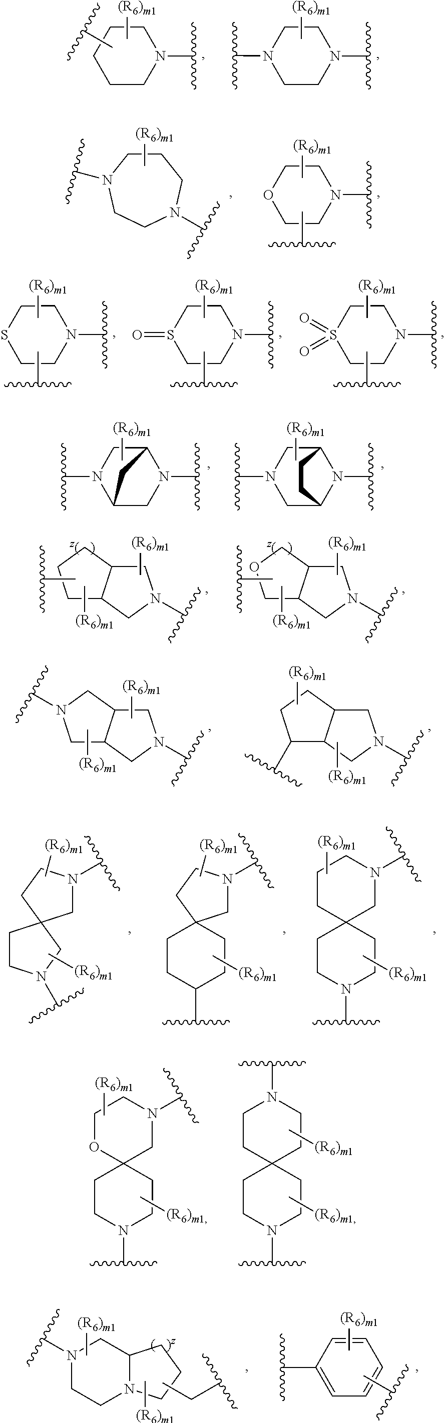

wherein the representation of L is not limited directionally left to right as is depicted, rather either the left side or the right side of L can be bound to the W₁ side of the compound of Formula I;

R₆ is independently —H, , —C₁-C₄ alkyl, gen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

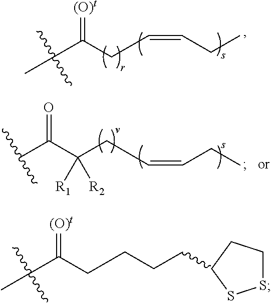

with the proviso that there is at least one

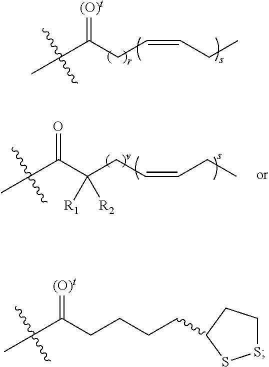

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, gen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

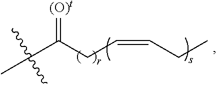

then t must be 0; and
when m, n, o, p, and q are each 0, and W₁ and W₂ are each null, then Z must not be

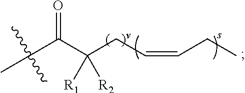

In another aspect, compounds of the Formula III are described:

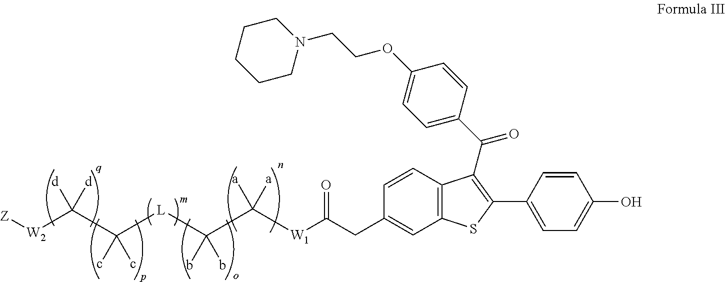

and pharmaceutically acceptable salts, hydrates, solvates, prodrugs, enantiomers and stereoisomers thereof;

wherein

W₁ and W₂ are each independently null, O, S, NH, NR, or W₁ and W₂ can be taken together can form an imidazolidine or piperazine group,

each a, b, c, and d is independently —H, , —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;

each n, o, p, and q is independently 0, 1 or 2;

L is independently null, —O—, —S—, —S(O)—, —S(O)₂—, —S—S—, —(C₁-C₆alkyl)-, —(C₃-C₆cycloalkyl)-, a heterocycle, a heteroaryl,

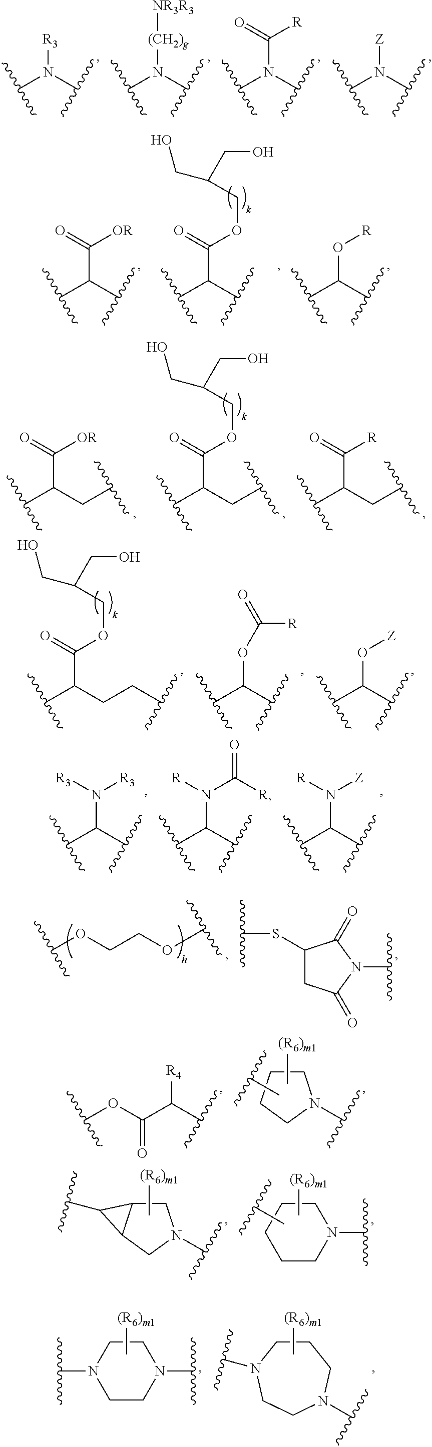

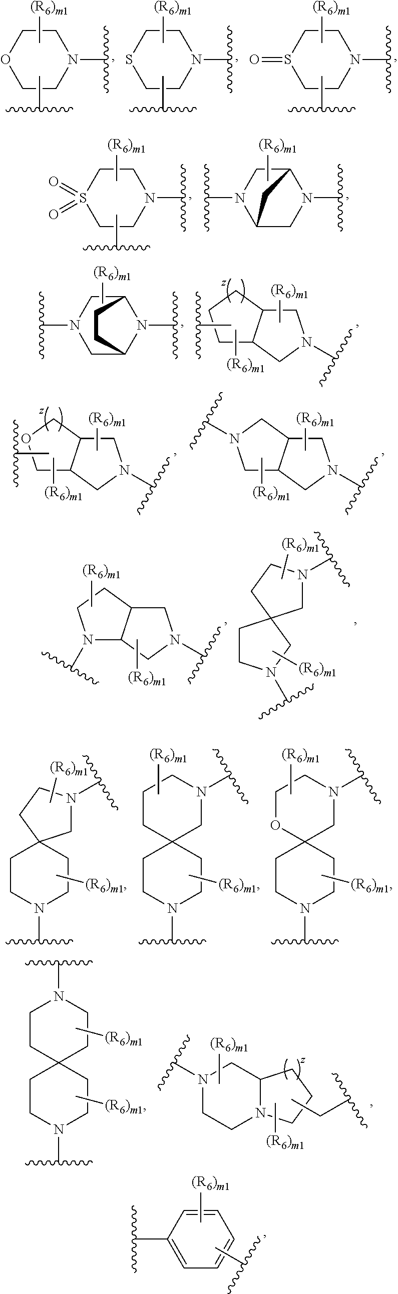

wherein the representation of L is not limited directionally left to right as is depicted, rather either the left side or the right side of L can be bound to the W₁ side of the compound of Formula I;

R₆ is independently —H, , —C₁-C₄ alkyl, gen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3; each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

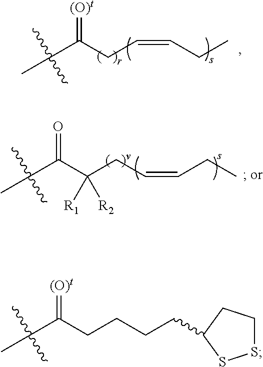

with the proviso that there is at least one

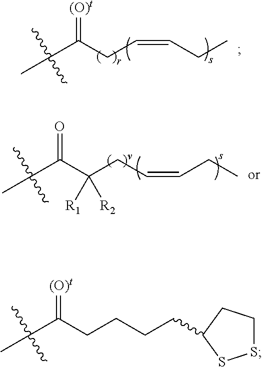

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, gen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen.

provided that
when m, n, o, p, and q are each 0, W₁ and W₂ are each null, and Z is

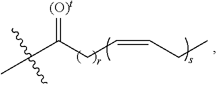

then t must be 0; and
when m, n, o, p, and q are each 0, and W₁ and W₂ are each null, then Z must not be

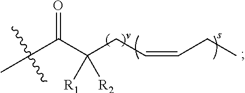

In Formula I, Formula II, and Formula III, any one or more of H may be substituted with a deuterium. It is also understood in Formula I, Formula II, and Formula III, that a methyl substituent can be substituted with a C₁-C₆ alkyl.

Also described are pharmaceutical formulations comprising at least one fatty acid raloxifene derivative.

Also described herein are methods of treating a disease susceptible to treatment with a fatty acid raloxifene derivative in a patient in need thereof by administering to the patient an effective amount of a fatty acid raloxifene derivative.

The invention also includes pharmaceutical compositions that comprise an effective amount of a fatty acid raloxifene derivative and a pharmaceutically acceptable carrier. The compositions are useful for treating osteoporosis or preventing invasive breast cancer. The invention includes a fatty acid raloxifene derivative provided as a pharmaceutically acceptable prodrug, a hydrate, a salt, such as a pharmaceutically acceptable salt, enantiomer, stereoisomer, or mixtures thereof.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### DETAILED DESCRIPTION OF THE INVENTION

Inflammation is believed to play an important part during the accelerated bone loss at menopause. As described earlier, omega-3 fatty acids such as DHA and EPA have been known to have anti-inflammatory, as well as anti-cancer properties. Raloxifene has been approved by the FDA for the treatment of osteoporosis and for the prevention of invasive breast cancer. The fatty acid raloxifene derivatives possess the ability to treat osteoporosis, endometriosis, uterine fibrosis, metabolic dyslipidemia, coronary heart disease and for the prevention of invasive breast cancer in postmenopausal women.

The fatty acid raloxifene derivatives have been designed to bring together raloxifene and omega-3 fatty acids into a single molecular conjugate. In addition, the Fatty Acid Raloxifene Derivatives have also been designed to bring together raloxifene and lipoic acid into a single molecular conjugate. The activity of the fatty acid raloxifene derivatives is substantially greater than the sum of the individual components of the molecular conjugate, suggesting that the activity induced by the fatty acid raloxifene derivatives is synergistic.

### DEFINITIONS

The following definitions are used in connection with the fatty acid raloxifene derivatives:

The term “fatty acid raloxifene derivatives” includes any and all possible isomers, stereoisomers, enantiomers, diastereomers, tautomers, pharmaceutically acceptable salts, hydrates, solvates, and prodrugs of the fatty acid raloxifene derivatives described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl. It is understood that any of the substitutable hydrogens on a cycloalkyl can be substituted with halogen, C₁-C₃ alkyl, hydroxyl, alkoxy and cyano groups.

The term “heterocycle” as used herein refers to a cyclic hydrocarbon containing 3-6 atoms wherein at least one of the atoms is an O, N, or S. Examples of heterocycles include, but are not limited to, aziridine, oxirane, thiirane, azetidine, oxetane, thietane, pyrrolidine, tetrahydrofuran, tetrahydrothiophene, piperidine, tetrahydropyran, thiane, imidazolidine, oxazolidine, thiazolidine, dioxolane, dithiolane, piperazine, oxazine, dithiane, and dioxane.

The term “heteroaryl” as used herein refers to a monocyclic or bicyclic ring structure having 5 to 12 ring atoms wherein one or more of the ring atoms is a heteroatom, e.g. N, O or S and wherein one or more rings of the bicyclic ring structure is aromatic. Some examples of heteroaryl are pyridyl, furyl, pyrrolyl, thienyl, thiazolyl, oxazolyl, imidazolyl, indolyl, tetrazolyl, benzofuryl, xanthenes and dihydroindole. It is understood that any of the substitutable hydrogens on a heteroaryl can be substituted with halogen, C₁-C₃ alkyl, hydroxyl, alkoxy and cyano groups.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “fatty acid” as used herein means an omega-3 fatty acid and fatty acids that are metabolized in vivo to omega-3 fatty acids. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid). In addition, the term “fatty acid” can also refer to medium chain fatty acids such as lipoic acid.

The term “raloxifene” as used herein means the molecule known as raloxifene and any derivative thereof.

A “subject” is a mammal, e.g., a human, mouse, rat, guinea pig, dog, cat, horse, cow, pig, or non-human primate, such as a monkey, chimpanzee, baboon or rhesus, and the terms “subject” and “patient” are used interchangeably herein.

The invention also includes pharmaceutical compositions comprising an effective amount of a fatty acid raloxifene derivative and a pharmaceutically acceptable carrier. The invention includes a fatty acid raloxifene derivative provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “metabolic disease” as used herein refers to disorders, diseases and syndromes involving dyslipidemia, and the terms metabolic disorder, metabolic disease, and metabolic syndrome are used interchangeably herein.

An “effective amount” when used in connection with a fatty acid raloxifene derivative is an amount effective for treating or preventing a metabolic disease.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a fatty acid raloxifene derivative.

The following abbreviations are used herein and have the indicated definitions: Boc and BOC are tert-butoxycarbonyl, Boc₂O is di-tert-butyl dicarbonate, CDI is 1,1′-carbonyldiimidazole, DCC is N,N′-dicyclohexylcarbodiimide, DIEA is N,N-diisopropylethylamine, DMAP is 4-dimethylaminopyridine, DOSS is sodium dioctyl sulfosuccinate, EDC and EDCI are 1-ethyl-3-(3-dimethylaminopropyl)carbodiimide hydrochloride, EtOAc is ethyl acetate, h is hour, HATU is 2-(7-aza-1H-benzotriazole-1-yl)-1,1,3,3-tetramethyluronium hexafluorophosphate, HPMC is hydroxypropyl methylcellulose, oxone is potassium peroxymonosulfate, Pd/C is palladium on carbon, TFA is trifluoroacetic acid, TGPS is tocopherol propylene glycol succinate, THF is tetrahydrofuran, and TNF is tumor necrosis factor.

### Compounds

Accordingly in one aspect, a molecular conjugate is described which comprises a raloxifene and at least one fatty acid covalently linked, wherein the fatty acid is selected from the group consisting of omega-3 fatty acids, fatty acids that are metabolized in vivo to omega-3 fatty acids, and lipoic acid, and the conjugate is capable of hydrolysis to produce free raloxifene and free fatty acid provided that the conjugate is not (9Z,12Z,15Z)-4-(6-((9Z,12Z,15Z)-octadeca-9,12,15-trienoyloxy)-3-(4-(2-(piperidin-1-yl)ethoxy)benzoyl)benzo[b]thiophen-2-yl)phenyl octadeca-9,12,15-trienoate.

In some embodiments, the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid, and lipoic acid. In other embodiments, the fatty acid is selected from eicosapentaenoic acid and docosahexaenoic acid. In some embodiments, the hydrolysis is enzymatic.

In another aspect, the present invention provides fatty acid raloxifene derivatives according to Formula I:

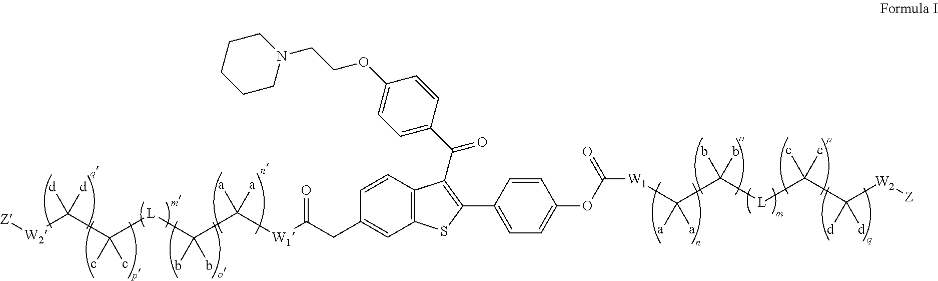

and pharmaceutically acceptable salts, hydrates, solvates, prodrugs, enantiomers and stereoisomers thereof;

wherein

W₁, W_{1′}, W_{2′}, W₂, a, b, c, d, e, k, m, m′, n, n′, o, o′, p, p′, q, q′, L, L′, Z, Z′, r, s, t, v, z, R₁, R₂, R₃, R₄, R and R₆ are as defined above for Formula I,

with the proviso that there is at least one

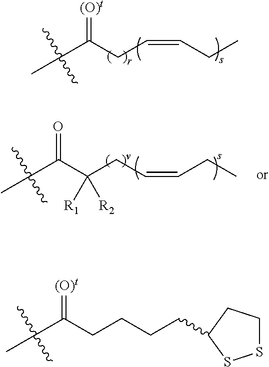

in the compound;

provided that
when m, n, o, p, and q are each 0, W₁ and W₂ are each null, and Z is

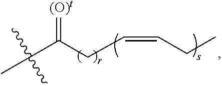

then t must be 0;
when m, n, o, p, and q are each 0, and W₁ and W₂ are each null, then Z must not be

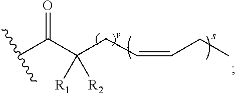

when m′, n′, o′, p′, and q′ are each 0, W_{1′} and W_{2′} are each null, and Z′ is

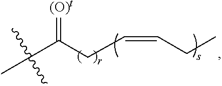

then t must be 0;
when m′, n′, o′, p′, and q′ are each 0, and W_{1′} and W_{2′} are each null, then Z′ must not be

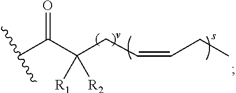

and
when m, m′, n, n′, o, o′, p, p′, q, and q′ are each 0, W₁, W₂, W_{1′} and W_{2′} are each null, each t is 1, Z and Z′ are each

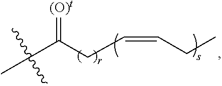

and each r is 7, then one s must be 5 or 6.

In some embodiments, one Z is

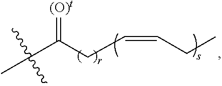

and r is 2.

In some embodiments, one Z is

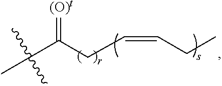

and r is 3.

In some embodiments, one Z is

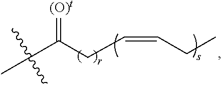

and r is 7.

In other embodiments, one Z is

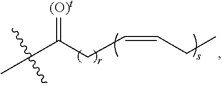

and s is 3.

In some embodiments, one Z is

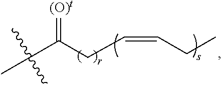

and s is 5.

In some embodiments, one Z is

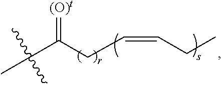

and s is 6.

In some embodiments, one Z is

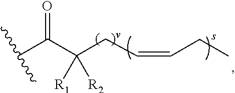

and v is 1.

In other embodiments, one Z is

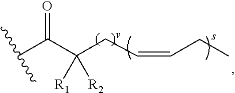

and v is 2.

In some embodiments, one Z is

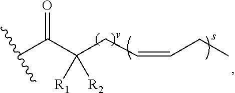

and v is 6.

In some embodiments, one Z is

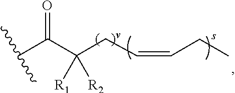

and s is 3.

In some embodiments, one Z is

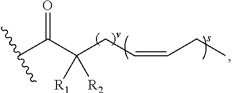

and s is 5.

In other embodiments, one Z is

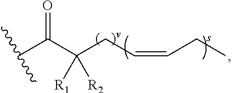

and s is 6.

In other embodiments, Z is

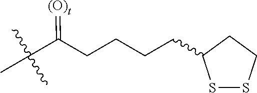

and t is 1.

In some embodiments, Z is

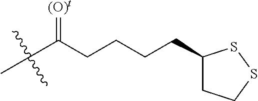

In some embodiments, W₁ is null, O, NH or N substituted with a C₁-C₆ alkyl.

In some embodiments, W_{1′} is null, O, NH or N substituted with a C₁-C₆ alkyl.

In some embodiments, W₂ is null, O, NH or N substituted with a C₁-C₆ alkyl.

In some embodiments, W_{2′} is null, O, NH or N substituted with a C₁-C₆ alkyl.

In some embodiments, each a and c is independently H, CH₃, —OCH₃, —OCH₂CH₃, or C(O)OR.

In some embodiments, m is 0.

In other embodiments, m is 1.

In other embodiments, m is 2.

In some embodiments, m′ is 0.

In other embodiments, m′ is 1.

In other embodiments, m′ is 2.

In some embodiments, each L is independently —S—, —S(O)—, —S(O)₂—, or —S—S—.

In some embodiments, each L′ is independently —S—, —S(O)—, —S(O)₂—, or —S—S—.

In some embodiments, each L is independently —O—,

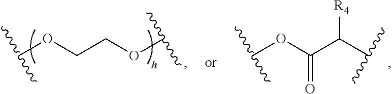

In some embodiments, each L′ is independently —O—,

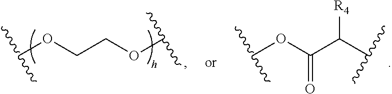

In some embodiments, each L is independently

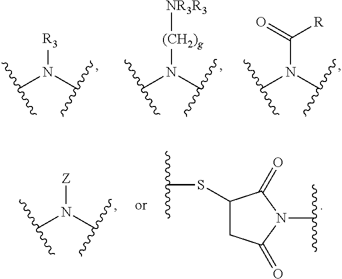

In some embodiments, each L′ is independently

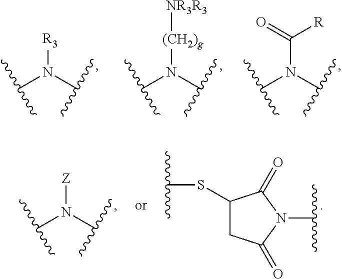

In some embodiments, each L is independently

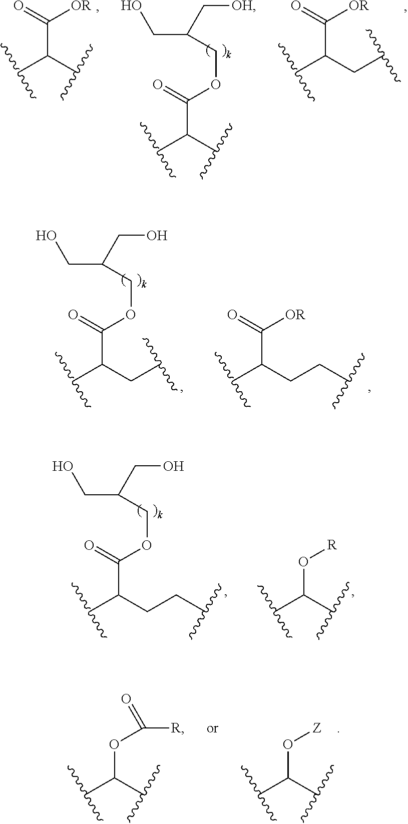

In some embodiments, each L′ is independently

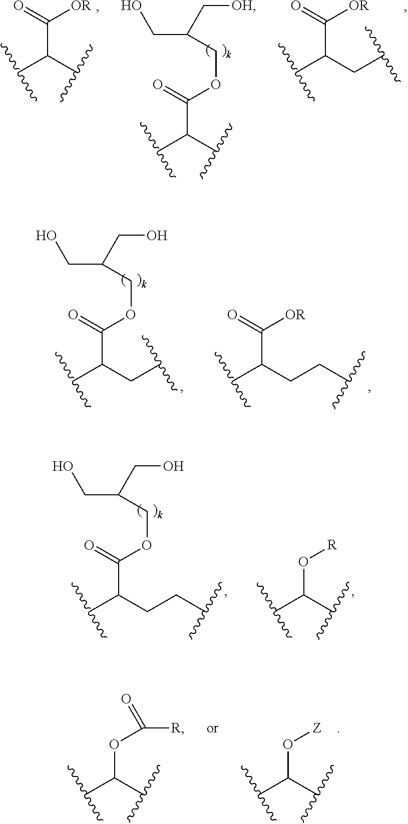

In some embodiments, each L is independently

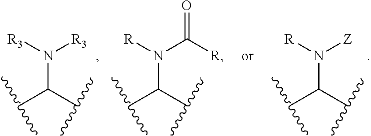

In some embodiments, each L′ is independently

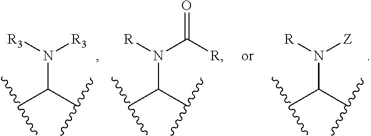

In some embodiments, L is

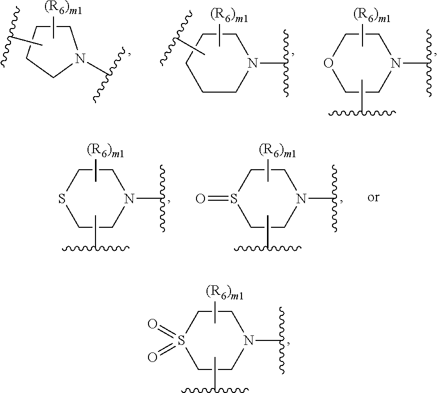

In some embodiments, L is

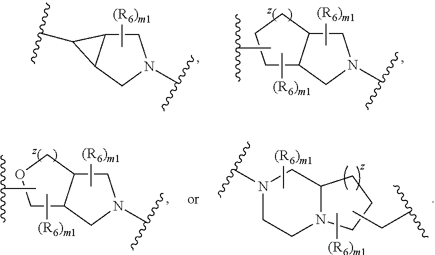

In some embodiments, L is

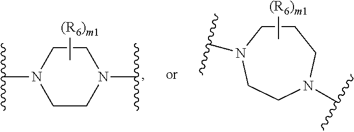

In some embodiments, L is

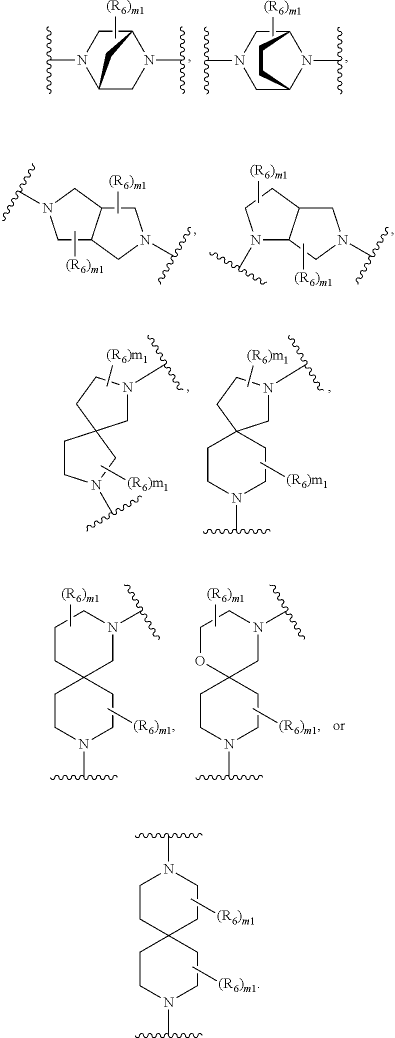

In some embodiments, L′ is

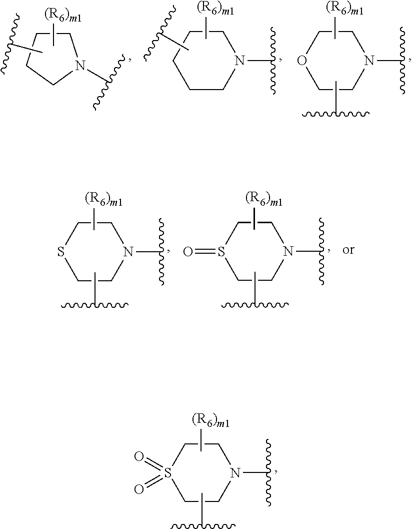

In some embodiments, L′ is

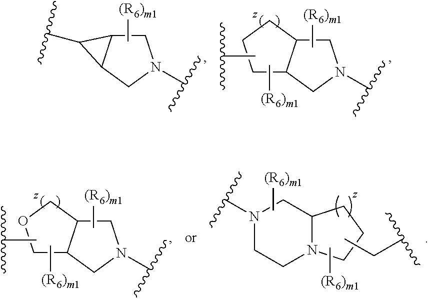

In some embodiments, L′ is

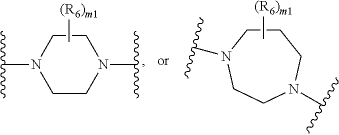

In some embodiments, L′ is

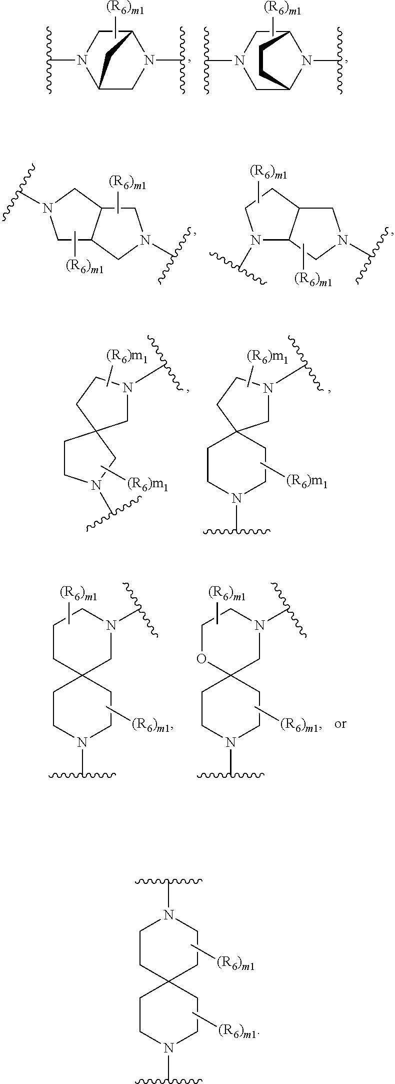

In some embodiments, one b is O—Z, Z is

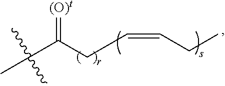

and t is 1.

In some embodiments, one d is C(O)OR.

In some embodiments n, o, p, and q are each 1.

In some embodiments n′, o′, p′, and q′ are each 1.

In some embodiments, two of n, o, p, and q are each 1.

In some embodiments, two of n′, o′, p′, and q′ are each 1.

In other embodiments, three of n, o, p, and q are each 1.

In other embodiments, three of n′, o′, p′, and q′ are each 1.

In other illustrative embodiments, compounds of Formula I are as set forth below:
4-(6-[(2S)-2-((4Z,7Z,10Z,13Z,16Z,19Z)docosa-4,7,10,13,16,19-hexaenoylamino)propanoyloxy]-3-{[4-(2-piperidylethoxy)phenyl]carbonyl}benzo[b]thiophen-2-yl)phenyl (2S)-2-((4Z,7Z,10Z,13Z,16Z,19Z)docosa-4,7,10,13,16,19-hexaenoylamino)propanoate (I-1);
4-(6-[(2S)-2-((4Z,7Z,10Z,13Z,16Z,19Z)docosa-4,7,10,13,16,19-hexaenoylamino)-3-methylbutanoyloxy]-3-{[4-(2-piperidylethoxy)phenyl]carbonyl}benzo[b]thiophen-2-yl)phenyl (2S)-2-((4Z,7Z,10Z,13Z,16Z,19Z)docosa-4,7,10,13,16,19-hexaenoylamino)-3-methylbutanoate (I-2);
4-(6-[3-((4Z,7Z,10Z,13Z,16Z,19Z)docosa-4,7,10,13,16,19-hexa enoylamino)propanoyloxy]-3-{[4-(2-piperidylethoxy)phenyl]carbonyl}benzo[b]thiophen-2-yl)phenyl 3-((4Z,7Z,10Z,13Z,16Z,19Z)docosa-4,7,10,13,16,19-hexaenoyl amino)propanoate (I-3);
(4Z,7Z,10Z,13Z,16Z,19Z)-N-(2-{[4-(6-{N-[2-((4Z,7Z,10Z,13Z, 16Z,19Z)docosa-4,7,10,13,16,19-hexaenoylamino)ethyl]carbamoyloxy}-3-{[4-(2-piperidylethoxy)phenyl]carbonyl}benzo[b]thiophen-2-yl)phenoxy]carbonylamino}ethyl)docosa-4,7,10,13,16,19-hexaenamide (I-4).

In another aspect, the present invention provides fatty acid raloxifene derivatives according to Formula II:

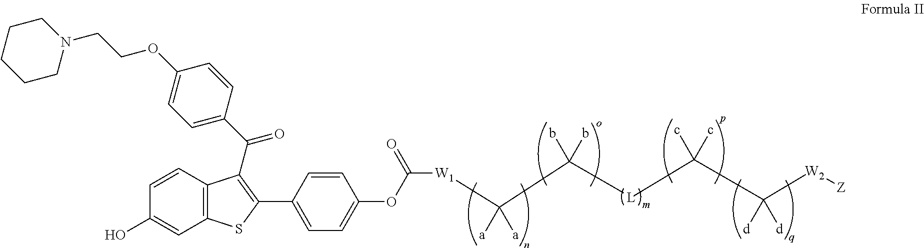

and pharmaceutically acceptable salts, hydrates, solvates, prodrugs, enantiomers and stereoisomers thereof;

wherein

W₁, W₂, a, b, d, e, k, m, n, o, p, q, L, Z, r, s, t, v, R₁, R₂, R₃, R₄, R and R₆ are as defined above for Formula II,

with the proviso that W₁ and W₂ can not be O simultaneously; and

with the proviso that there is at least one

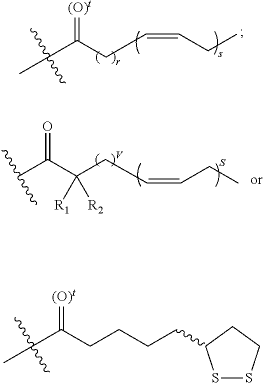

in the compound.

In some embodiments, one Z is

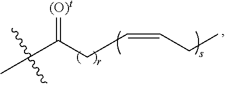

and r is 2.

In some embodiments, one Z is

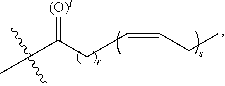

and r is 3.

In some embodiments, one Z is

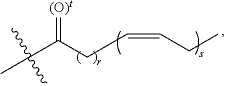

and r is 7.

In other embodiments, one Z is

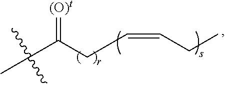

and s is 3.

In some embodiments, one Z is

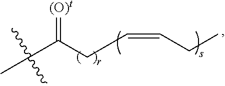

and s is 5.

In some embodiments, one Z is

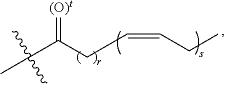

and s is 6.

In some embodiments, one Z is

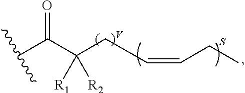

and v is 1.

In other embodiments, one Z is

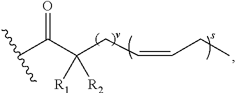

and v is 2.

In some embodiments, one Z is

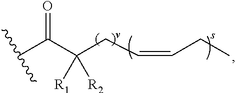

and v is 6.

In some embodiments, one Z is

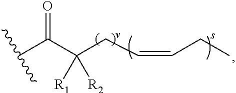

and s is 3.

In some embodiments, one Z is

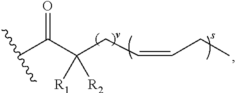

and s is 5.

In other embodiments, one Z is

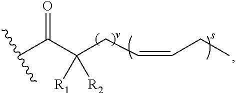

and s is 6.

In other embodiments, Z is

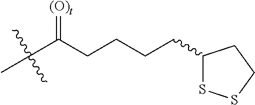

and t is 1.

In some embodiments, Z is

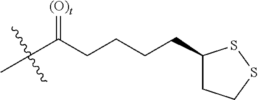

and t is 1.

In some embodiments, W₁ is null, O, NH or N substituted with a C₁-C₆ alkyl.

In some embodiments, W₂ is null, O, NH or N substituted with a C₁-C₆ alkyl.

In some embodiments, each a and c is independently H, CH₃, —OCH₃, —OCH₂CH₃, or C(O)OR.

In some embodiments, m is 0.

In other embodiments, m is 1.

In other embodiments, m is 2.

In some embodiments, each L is independently —S—, —S(O)—, —S(O)₂—, or —S—S—.

In some embodiments, each L is independently —O—,

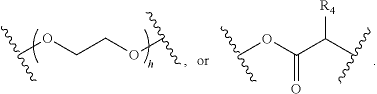

In some embodiments, each L is independently

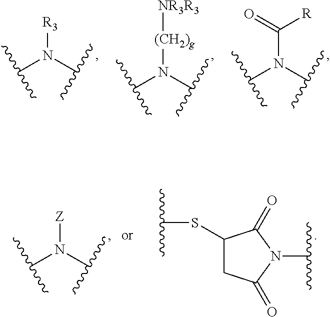

In some embodiments, each L is independently

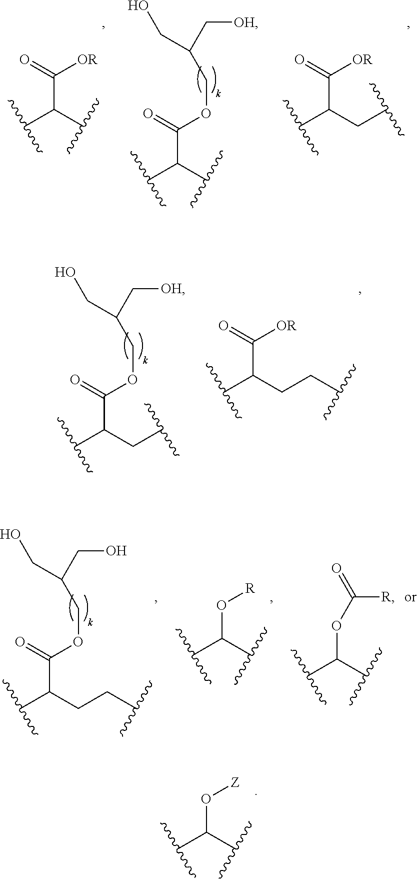

In some embodiments, each L is independently

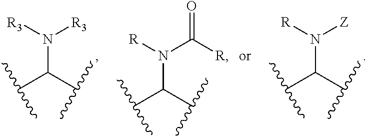

In some embodiments, L is

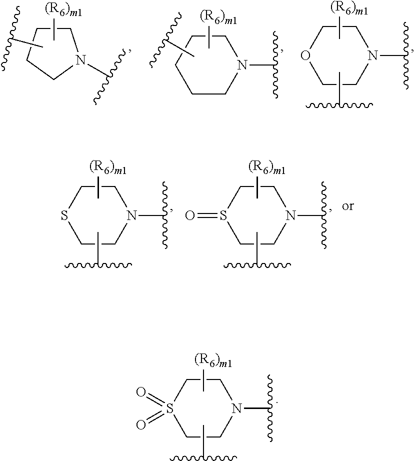

In some embodiments, L is

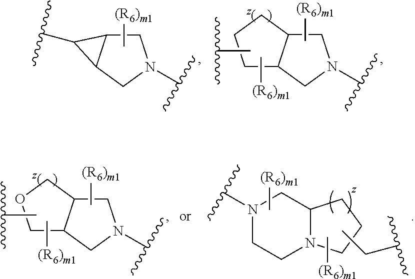

In some embodiments, L is

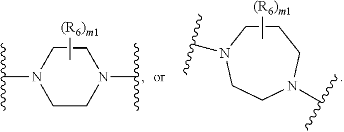

In some embodiments, L is

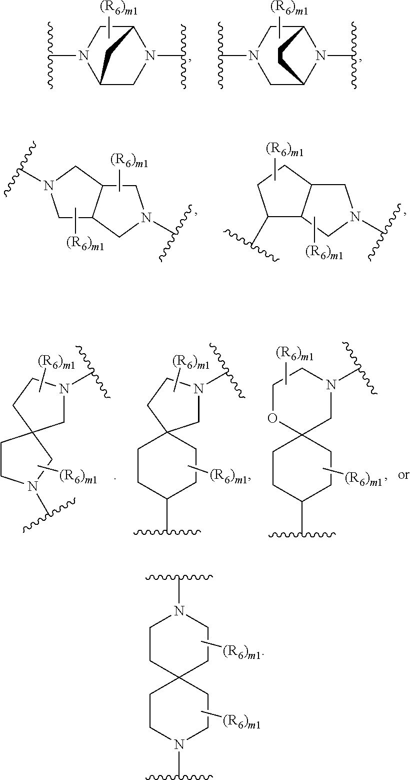

In some embodiments, one b is O—Z, Z is

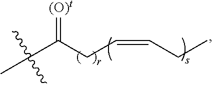

and t is 1.

In some embodiments, one d is C(O)OR.

In some embodiments, n, o, p, and q are each 1.

In some embodiments, two of n, o, p, and q are each 1.

In other embodiments, three of n, o, p, and q are each 1.

In some embodiments, t is 1.

In other illustrative embodiments, compounds of Formula II are as set forth below:
(S)-4-(6-hydroxy-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)propanoate (II-1);
(S)-4-(6-hydroxy-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)-3-methylbutanoate (II-2);
4-(6-hydroxy-3-(4-(2-morpholinoethoxy)benzoyl)benzo[b]thiophen-2-yl)phenyl 3-(4Z,7Z,10Z,13Z,16Z,19Z)-docosa-4,7,10,13,16,19-hexaenamidopropanoate (II-3);
4-(6-hydroxy-3-(4-(2-morpholinoethoxy)benzoyl)benzo[b]thiophen-2-yl)phenyl 2-(4Z,7Z,10Z,13Z,16Z,19Z)-docosa-4,7,10,13,16,19-hexaenamidoethylcarbamate (II-4).

In another aspect, the present invention provides fatty acid raloxifene derivatives according to Formula III:

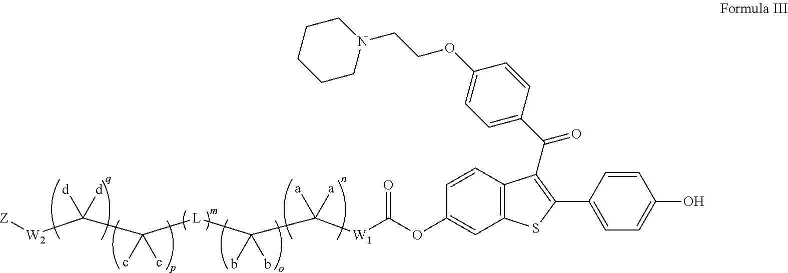

and pharmaceutically acceptable salts, hydrates, solvates, prodrugs, enantiomers and stereoisomers thereof;

wherein

W₁, W₂, a, c, d, e, k, m, n, o, p, q, L, Z, r, s, t, v, R₁, R₂, R₃, R₄, R and R₆ are as defined above for Formula III,

with the proviso that W₁ and W₂ can not be O simultaneously; and

with the proviso that there is at least one

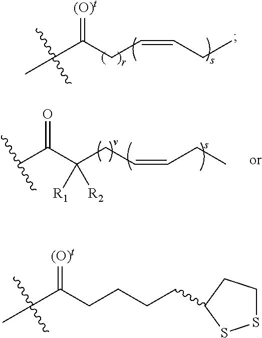

in the compound.

In some embodiments, one Z is

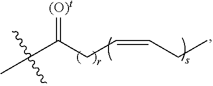

and r is 2.

In some embodiments, one Z is

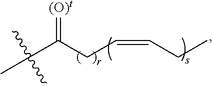

and r is 3.

In some embodiments, one Z is

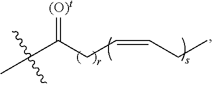

and r is 7.

In other embodiments, one Z is

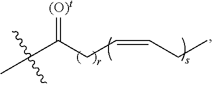

and s is 3.

In some embodiments, one Z is

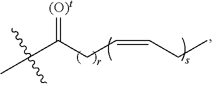

and s is 5.

In some embodiments, one Z is

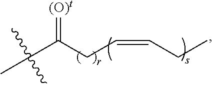

and s is 6.

In some embodiments, one Z is

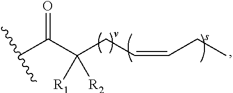

and v is 1.

In other embodiments, one Z is

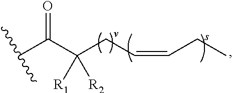

and v is 2.

In some embodiments, one Z is

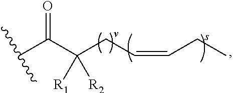

and v is 6.

In some embodiments, one Z is

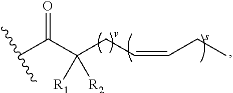

and s is 3.

In some embodiments, one Z is

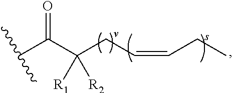

and s is 5.

In other embodiments, one Z is

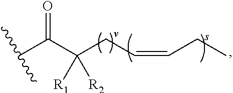

and s is 6.

In other embodiments, Z is

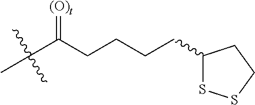

and t is 1.

In some embodiments, Z is

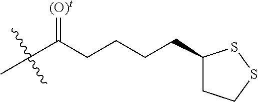

and t is 1.

In some embodiments, W₁ is null, O, NH or N substituted with a C₁-C₆ alkyl.

In some embodiments, W₂ is null, O, NH or N substituted with a C₁-C₆ alkyl.

In some embodiments, each a and c is independently H, CH₃, —OCH₃, —OCH₂CH₃, or C(O)OR.

In some embodiments, m is 0.

In other embodiments, m is 1.

In other embodiments, m is 2.

In some embodiments, each L is independently —S—, —S(O)—, —S(O)₂—, or —S—S—.

In some embodiments, each L is independently —O—,

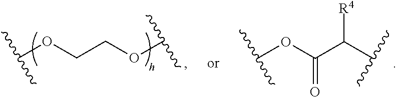

In some embodiments, each L is independently

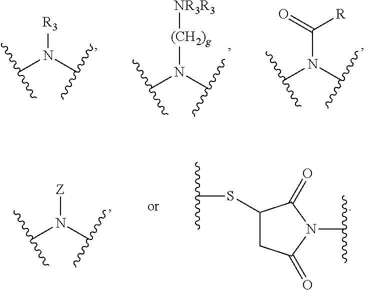

In some embodiments, each L is independently

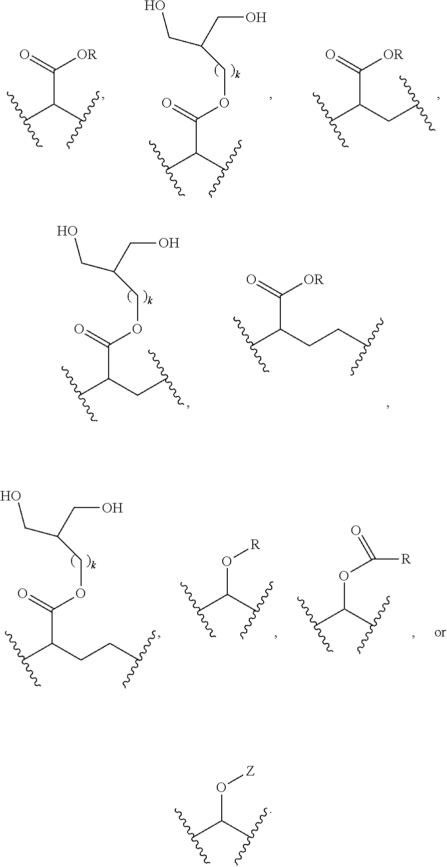

In some embodiments, each L is independently

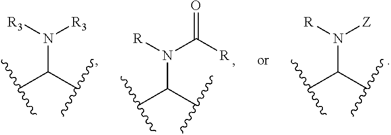

In some embodiments, L is

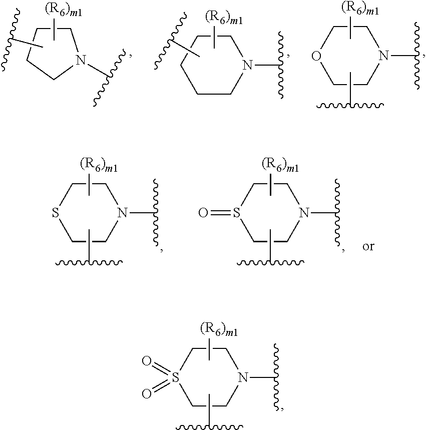

In some embodiments, L is

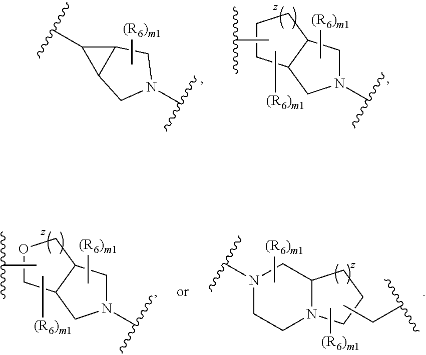

In some embodiments, L is

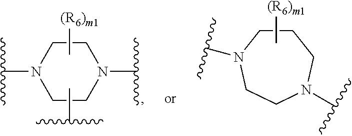

In some embodiments, L is

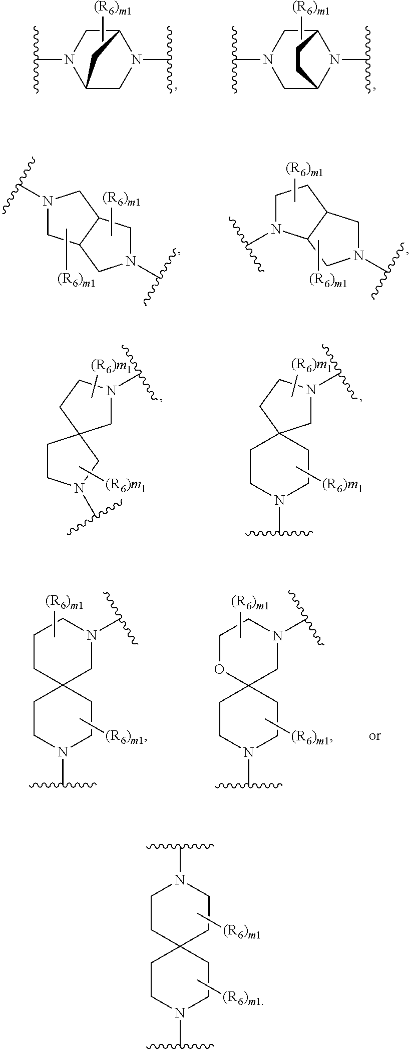

In some embodiments, one b is O—Z, Z is

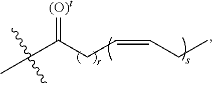

and t is 1.

In some embodiments, one d is C(O)OR.

In some embodiments, n, o, p, and q are each 1.

In some embodiments, two of n, o, p, and q are each 1.

In other embodiments, three of n, o, p, and q are each 1.

In some embodiments, t is 1.

In other illustrative embodiments, compounds of Formula III are as set forth below:
(S)-2-(4-hydroxyphenyl)-3-(4-(2-morpholinoethoxy)benzoyl)benzo[b]thiophen-6-yl 2-((4Z,7Z,10Z,13Z,16Z,19Z)-docosa-4,7,10,13,16,19-hexaenamido)propanoate (III-1);
(S)-2-(4-hydroxyphenyl)-3-(4-(2-morpholinoethoxy)benzoyl)benzo[b]thiophen-6-yl 2-((4Z,7Z,10Z,13Z,16Z,19Z)-docosa-4,7,10,13,16,19-hexaenamido)-3-methylbutanoate (III-2);
2-(4-hydroxyphenyl)-3-(4-(2-morpholinoethoxy)benzoyl)benzo[b]thiophen-6-yl 3-(4Z,7Z,10Z,13Z,16Z,19Z)-docosa-4,7,10,13,16,19-hexaenamidopropanoate (III-3);
2-(4-hydroxyphenyl)-3-(4-(2-morpholinoethoxy)benzoyl)benzo[b]thiophen-6-yl 2-(4Z,7Z,10Z,13Z,16Z,19Z)-docosa-4,7,10,13,16,19-hexaenamidoethylcarbamate (III-4).

In Formula I, Formula II, and Formula III, any one or more of H may be substituted with a deuterium. It is also understood in Formula I, Formula II and Formula III, that a methyl substituent can be substituted with a C₁-C₆ alkyl.

### Methods for Using Fatty Acid Raloxifene Derivatives

Provided herein are methods for treating osteoporosis, endometriosis, uterine fibrosis, metabolic dyslipidemia, coronary heart disease and for preventing invasive breast cancer in postmenopausal women.

Since the compounds of the invention are antiestrogens and antiandrogens, they can be used for antiestrogen and antiandrogen therapy. Described herein are methods for treating mammary and prostatic tumors or benign prostatic hypertrophy and treating and preventing mammary and prostatic fibrocystic disease.

In some embodiments, the subject is administered an effective amount of a fatty acid raloxifene derivative.

Administration of the fatty acid raloxifene derivatives can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a fatty acid raloxifene derivative and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the fatty acid raloxifene derivative is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the fatty acid raloxifene derivatives.

The fatty acid raloxifene derivatives can be also formulated as a suppository that can be prepared from fatty emulsions or suspensions; using polyalkylene glycols such as propylene glycol, as the carrier.

The fatty acid raloxifene derivatives can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the contents of which are hereby incorporated in their entirety.

Fatty acid raloxifene derivatives can also be delivered by the use of monoclonal antibodies as individual carriers to which the fatty acid raloxifene derivatives are coupled. The fatty acid raloxifene derivatives can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the fatty acid raloxifene derivatives can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, fatty acid raloxifene derivatives are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 90%, from about 10% to about 90%, or from about 30% to about 90% of the fatty acid raloxifene derivative by weight or volume.

The dosage regimen utilizing the fatty acid raloxifene derivative is selected in accordance with a variety of factors including type, species, age, weight, sex and medical condition of the patient; the severity of the condition to be treated; the route of administration; the renal or hepatic function of the patient; and the particular fatty acid raloxifene derivative employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 20 mg to about 5,000 mg of the fatty acid raloxifene derivative per day. Compositions for in vivo or in vitro use can contain about 20, 50, 75, 100, 150, 250, 500, 750, 1,000, 1,250, 2,500, 3,500, or 5,000 mg of the fatty acid raloxifene derivative. In one embodiment, the compositions are in the form of a tablet that can be scored. Effective plasma levels of the fatty acid raloxifene derivative can range from about 5 ng/mL to about 5,000 ng/mL. Appropriate dosages of the fatty acid raloxifene derivatives can be determined as set forth in Goodman, L. S.; Gilman, A. The Pharmacological Basis of Therapeutics, 5th ed.; MacMillan: New York, 1975, pp. 201-226.

Fatty acid raloxifene derivatives can be administered in a single daily dose, or the total daily dosage can be administered in divided doses of two, three or four times daily. Furthermore, fatty acid raloxifene derivatives can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the fatty acid raloxifene derivative ranges from about 0.1% to about 15%, w/w or w/v.

### Methods of Making

### Methods for Making the Fatty Acid Raloxifene Derivatives

Examples of synthetic pathways useful for making fatty acid raloxifene derivatives of Formula I, Formula II and Formula III are set forth in the Examples below and generalized in Schemes 1-11.

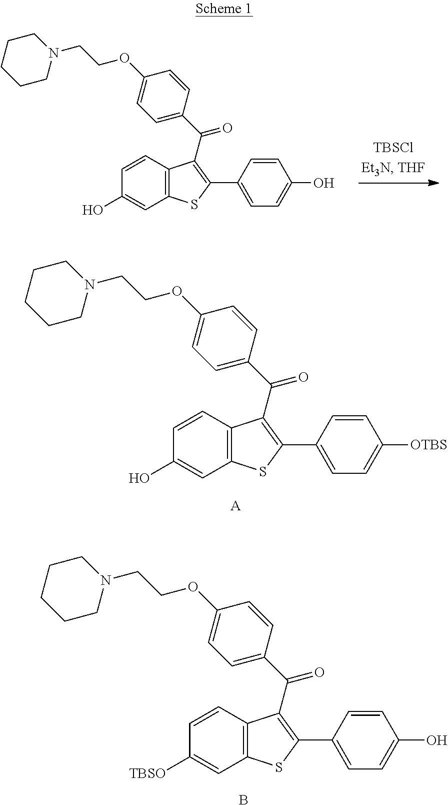

Raloxifene can be mono-silylated with a TBS group using TBSCl and triethylamine in a solvent such as THF according to the protocols detailed in Grese et al. J. Med. Chem. 1997, 40, 146-167. To those skilled in the art, the TBS group is a protecting group for the phenol residue and can be removed by treatment with n-Bu₄NF or with acids such as HCl or TFA. The silylation procedure affords a mixture of compounds A and B, which can be separated by chromatography techniques. Compound A is used to generate compounds of Formula III and compound B is used to generate compounds of Formula II.

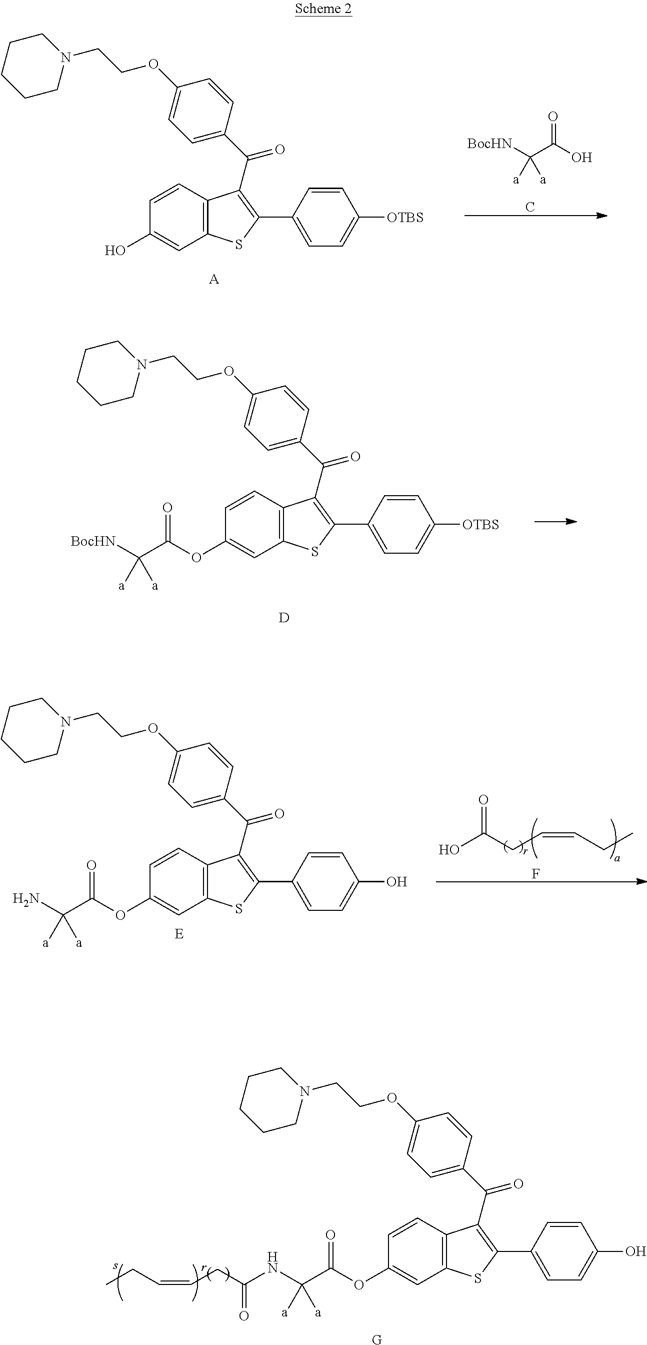

wherein a, r, and s are as defined above.

Compound A is coupled with compound C using EDCI or HATU in the presence of DIEA to afford compound D. Treatment of D with acids such as TFA or HCl removes both the BOC and TBS groups to afford compound E. Compound E, in turn, can be coupled with fatty acids of formula F to afford compounds of formula G. To those familiar in the art, the fatty acid of formula F can also be lipoic acid.

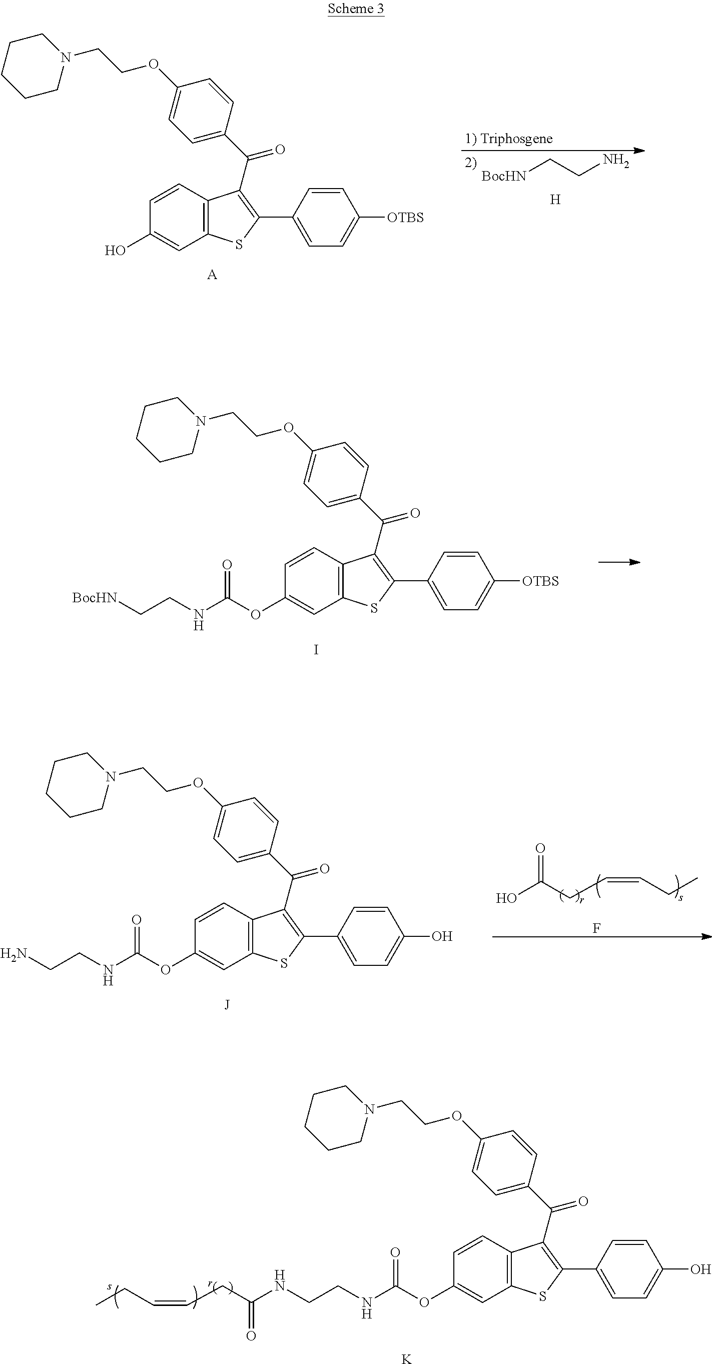

wherein r and s are as defined above.

Compound A can be reacted first with triphosgene, followed by the addition of compound H to afford compound I. Treatment of I with acids such as HCl or TFA affords compound J. Compound J can be coupled with a fatty acid of the formula F using either EDCI or HATU in the presence of DIEA to afford compounds of the formula K.

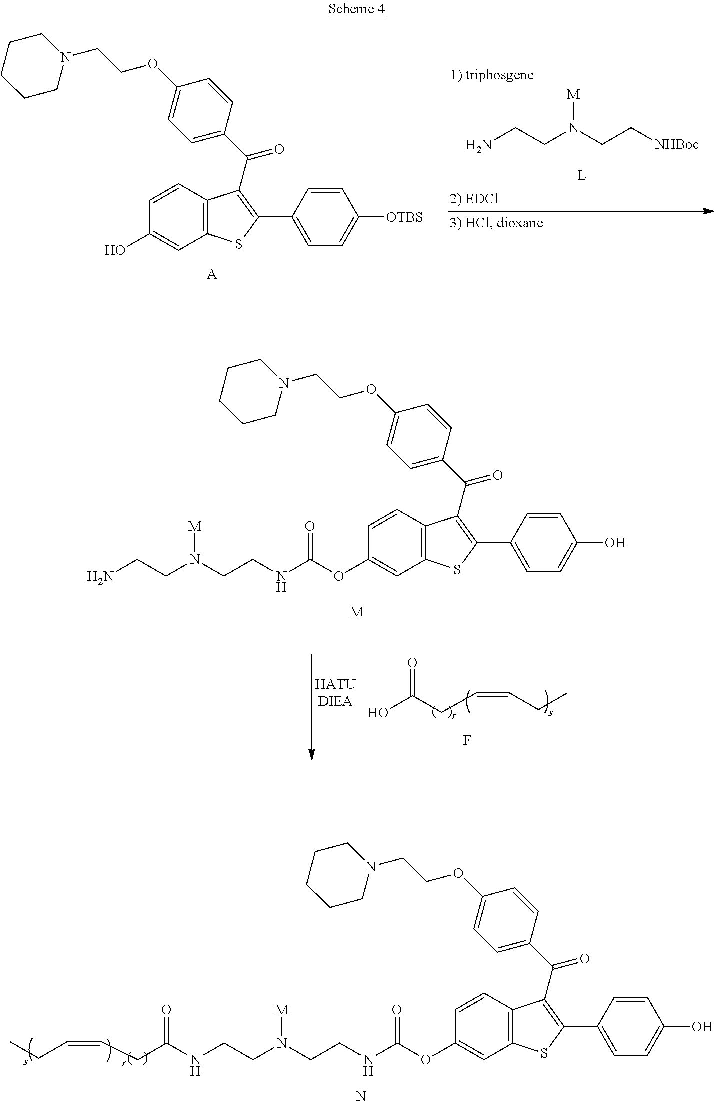

wherein M is R₃ or C(O)R₃, and R₃, r, and s are as defined above.

In formula L, when M=R₃, the corresponding amine can be obtained from commercial sources or prepared according to the procedures outlined in Krapcho et al. Synthetic Commun. 1990, 20, 2559-2564. In formula L, when M=C(O)R₃, the corresponding acylated amine can be prepared using the procedures outlined in Andruszkiewicz et al. Synthetic Commun. 2008, 38, 905-913. Compound A is reacted first with triphosgene, followed by the addition of the amine L. The resulting carbamate derivative can be treated with acids such as TFA or HCl in a solvent such as CH₂Cl₂ or dioxane to produce compounds of the formula M. Activation of compound M with a coupling agent such as HATU in the presence of an amine such as DIEA followed by addition of a fatty acid of formula F affords compounds of the formula N.

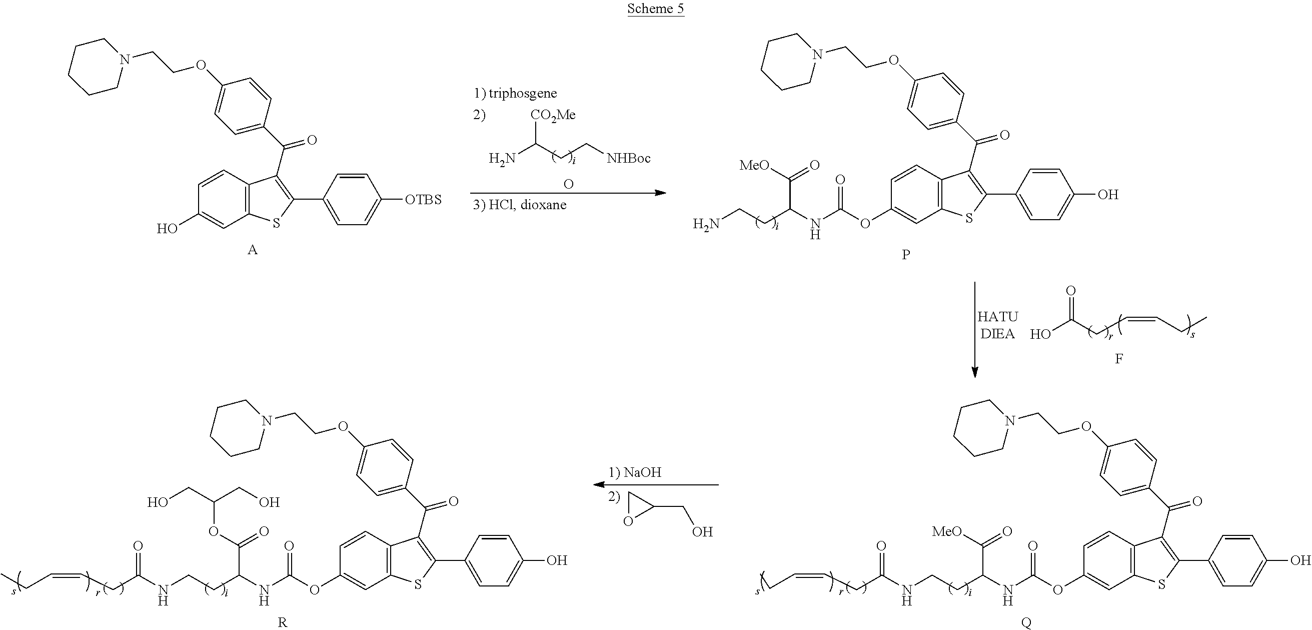

wherein r and s are as defined above.

Compound A can be treated with triphosgene, followed by the addition of the amine of the formula 0 (where i=0, 1, 2 or 3). Treatment with acids such as TFA or HCl in a solvent such as CH₂Cl₂ or dioxane remove the BOC and TBS protecting groups to produce the coupled compound P. Activation of compound P with a coupling agent such as HATU in the presence of an amine such as DIEA followed by addition of a fatty acid of formula F affords compounds of the formula Q. Hydrolysis of the ester under basic conditions such as NaOH or LiOH produces the corresponding acid, which can be coupled with glycidol to afford compounds of the formula R.

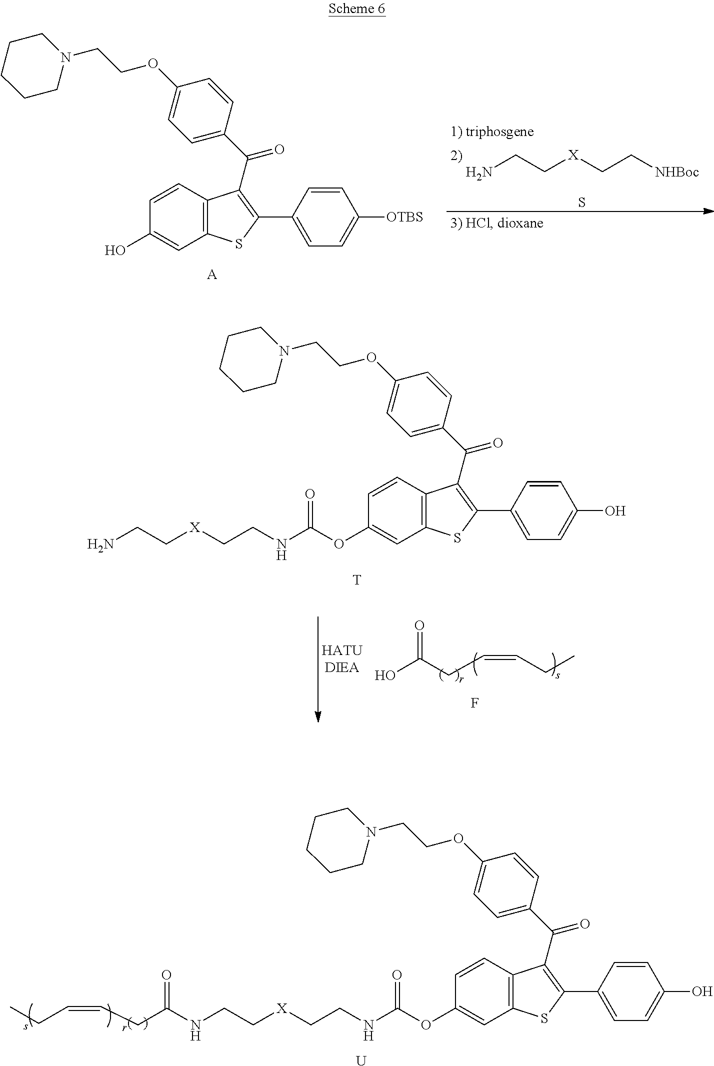

wherein X is S, S—S or O, and r and s are as defined above.

In formula S, when X═S, the corresponding amine could be obtained from commercial sources. In formula S, when X═S—S, the corresponding amine could be prepared according to the procedure outlined in Jacobson, K. et al. Bioconjugate Chem. 1995, 6, 255-263. In formula S, when X═O, the corresponding amine could be prepared according to the procedure outlined in Dahan et al. J. Org. Chem. 2007, 72, 2289-2296. Compound A can be treated with first with triphosgene, followed by the addition of the desired amine S. The resulting compound can be treated with acids such as TFA or HCl in a solvent such as CH₂Cl₂ or dioxane remove the BOC and TBS protecting groups to produce the coupled compound T. Activation of compound T with a coupling agent such as HATU in the presence of an amine such as DIEA followed by addition of a fatty acid of formula F affords compounds of the formula U. To those skilled in the art, the sulfur group in formula U can be oxidized to the corresponding sulfoxide or sulfone using an oxidizing agent such as H₂O₂ or oxone.

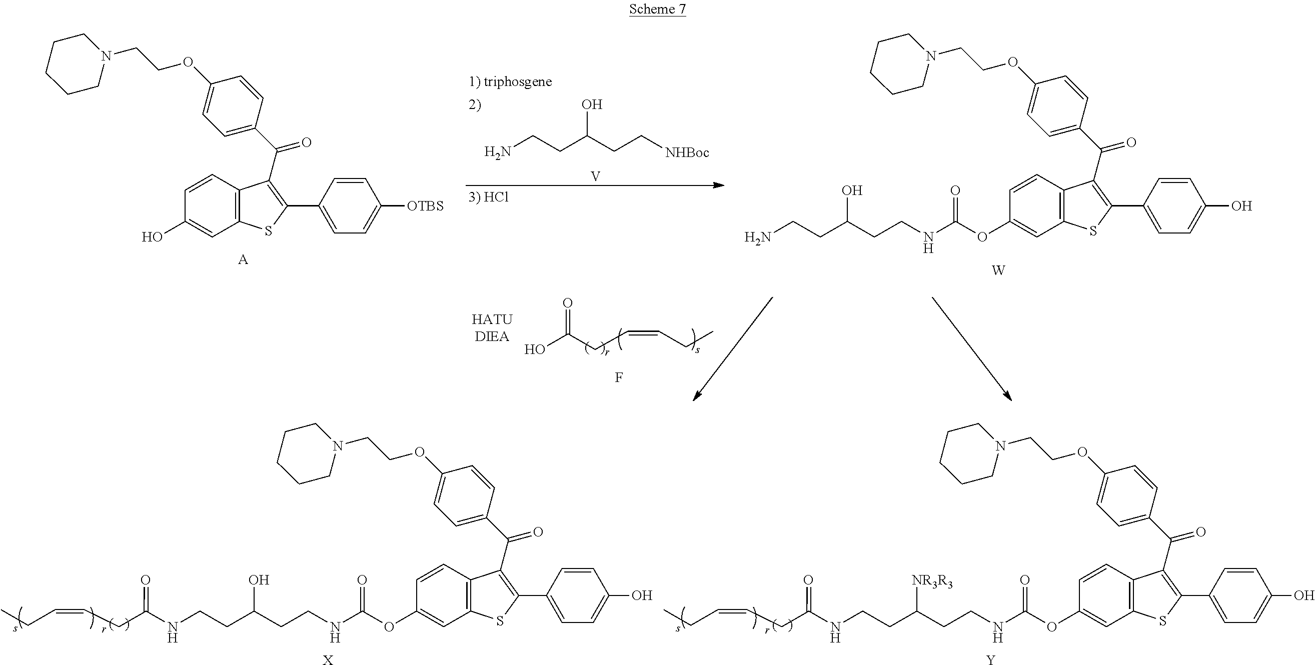

wherein R₃, r, and s are as defined above.

The amine V can be prepared from the commercially available diamine according to the procedures outlined in Dahan et al. J. Org. Chem. 2007, 72, 2289-2296. Compound A can be treated with first with triphosgene, followed by the addition of the desired amine V. The resulting compound can be treated with acids such as TFA or HCl in a solvent such as CH₂Cl₂ or dioxane remove the BOC and TBS protecting groups to produce the coupled compound W. Activation of compound W with a coupling agent such as HATU in the presence of an amine such as DIEA followed by addition of a fatty acid of formula F affords compounds of the formula X. To those skilled in the art, the hydroxyl group can be further acylated or converted to an amino group by standard mesylation chemistry followed by displacement with sodium azide and hydrogenation over a catalyst such as Pd/C. The amine can be further acylated or alkylated, followed by the removal of the BOC group. The resulting amine can be coupled with a fatty acid of the formula F to afford compounds of the formula Y.

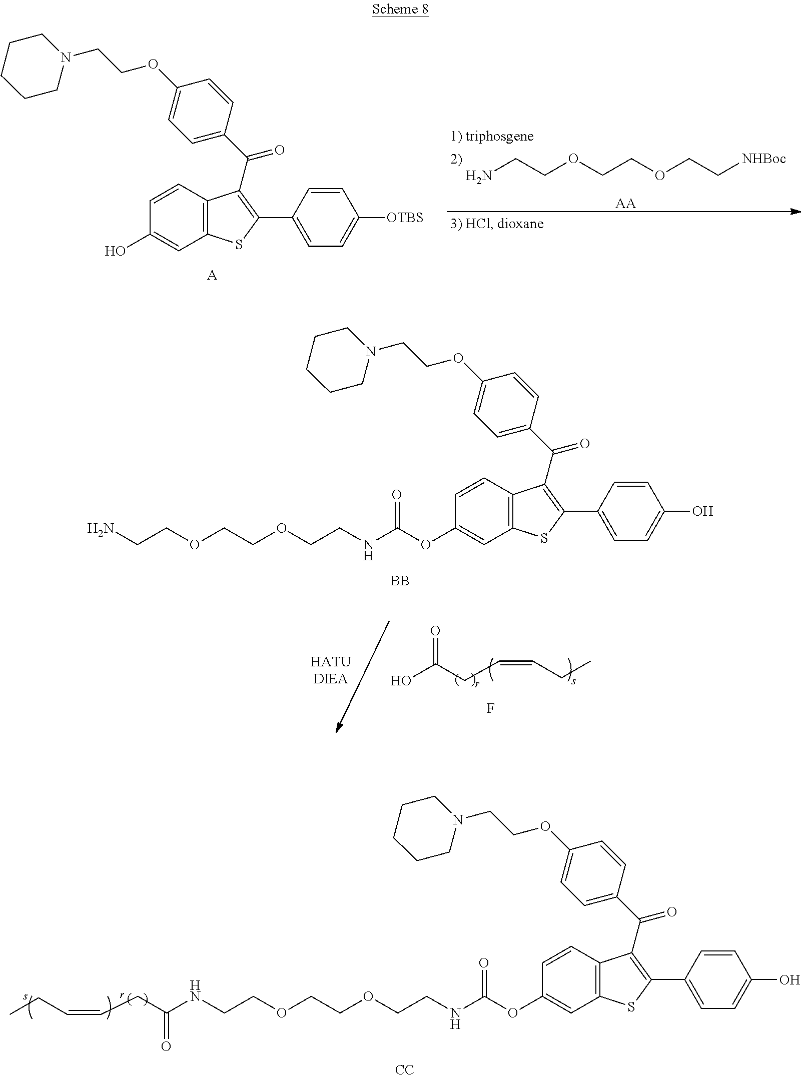

wherein r and s are as defined above.

Compound A can be treated with first with triphosgene, followed by the addition of the commercially available amine AA. The resulting compound can be treated with acids such as TFA or HCl in a solvent such as CH₂Cl₂ or dioxane remove the BOC and TBS protecting groups to produce the coupled compound BB. The resulting amine can be coupled with a fatty acid of the formula F using a coupling agent such as HATU in the presence of an amine such as DIEA to afford compounds of the formula CC.

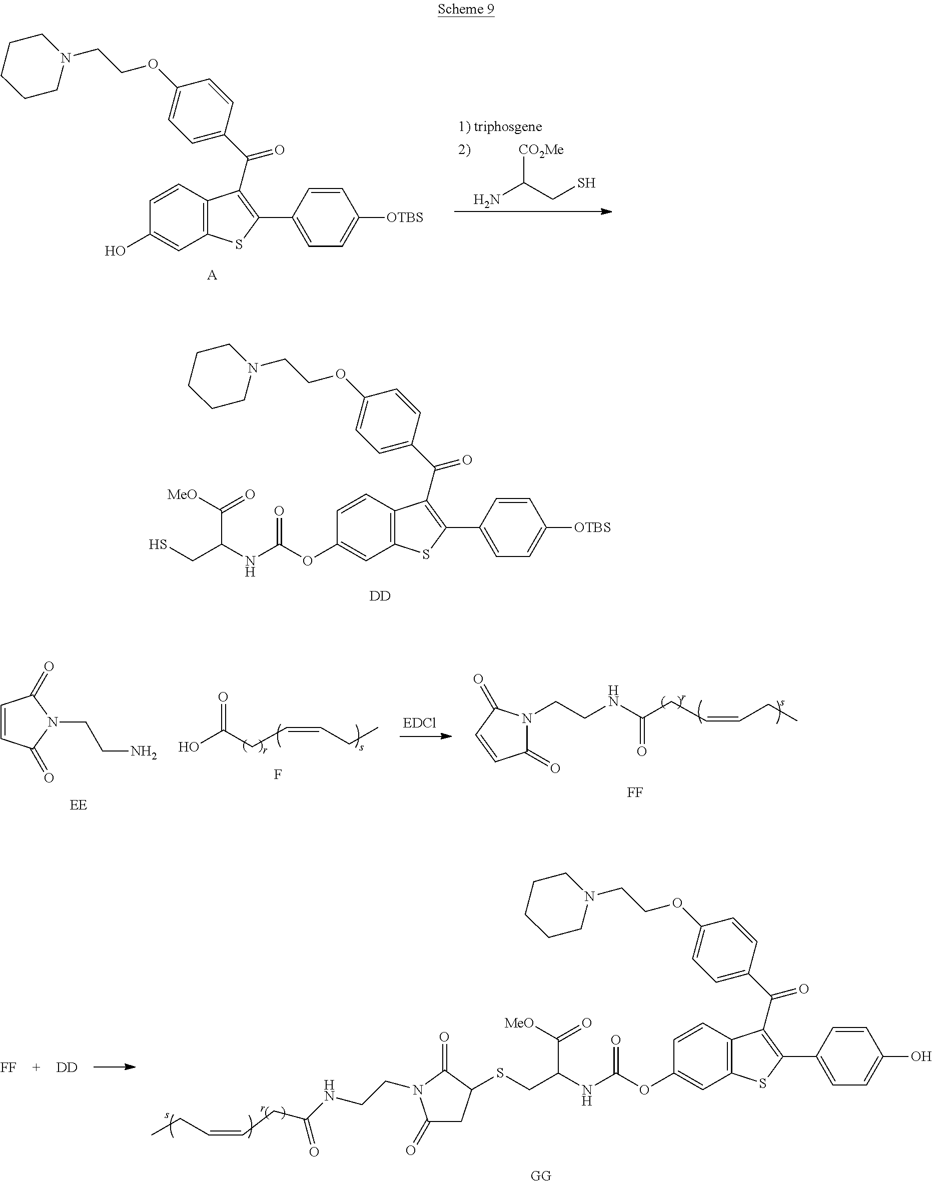

wherein r and s are as defined above.

Compound A can be treated with triphosgene, followed by the addition of the commercially available cysteine methyl ester to afford compound DD. The commercially available maleimide derivative EE can be coupled with a fatty acid of the formula F using a coupling agent such as HATU or EDCI to afford compounds of the formula FF. Compound FF can be coupled to compounds of the formula CC in a solvent such as acetonitrile, followed by treatment with acids such as TFA or HCl to afford compounds of the formula GG.

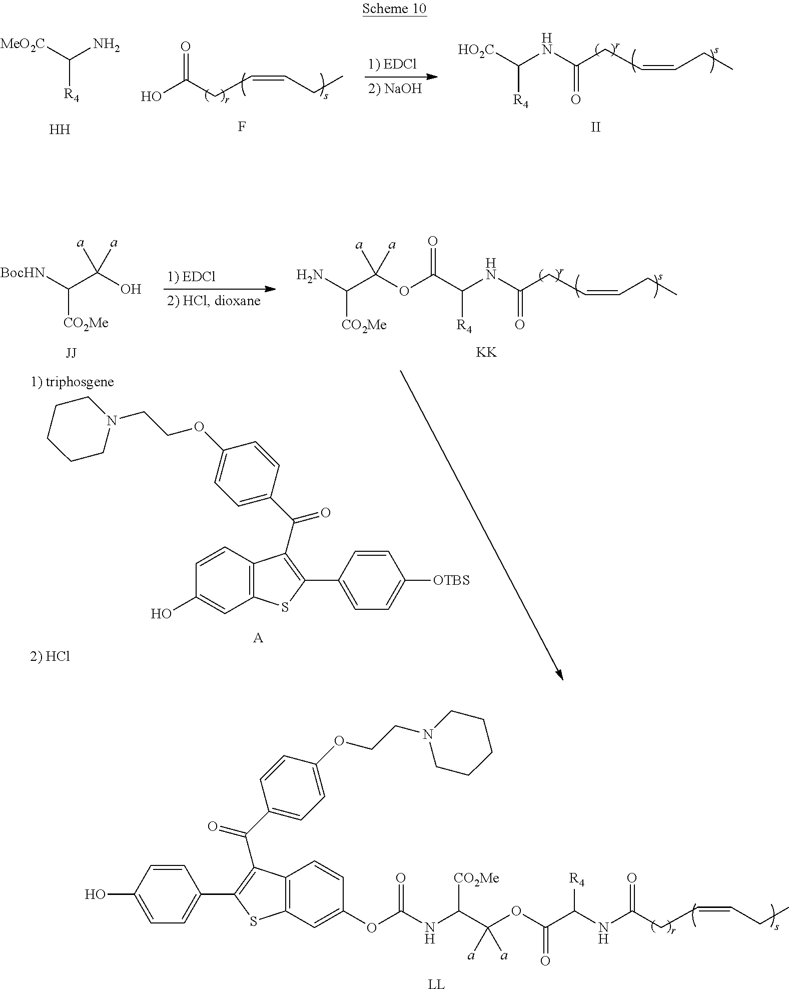

wherein R₄, a, r, and s are as defined above.

The commercially available amino acid esters HH can be coupled with a fatty acid of the formula F using a coupling agent such as EDCI or HATU, followed by alkaline hydrolysis of the methyl ester to afford compounds of the formula II. Compounds of the formula FF can be coupled with the commercially available BOC-amino acid derivatives JJ using a coupling agent such as EDCI or HATU. The BOC group can be removed by treatment with acids such as TFA or HCl to afford compounds of the formula KK. Compound A can be treated with triphosgene, followed by the addition of compound KK and treatment with acids such as TFA or HCl to afford compounds of the formula LL.

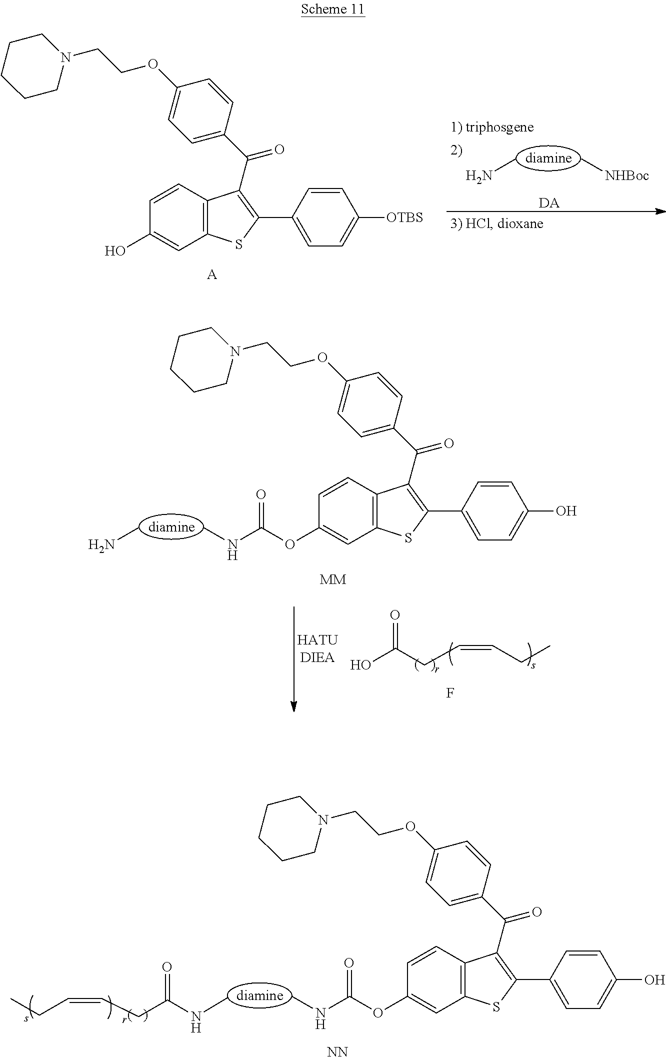

Compound A can be reacted first with triphosgene, followed by the addition of mono-Boc protected diamine DA followed by treatment with acids such as HCl or TFA affords compound MM. Compound MM can be coupled with a fatty acid of the formula F using either EDCI or HATU in the presence of DIEA to afford compounds of the formula NN. A variety of BOC-protected diamines are commercially available. The following diamines can be prepared according to the procedures outlined in the corresponding references:

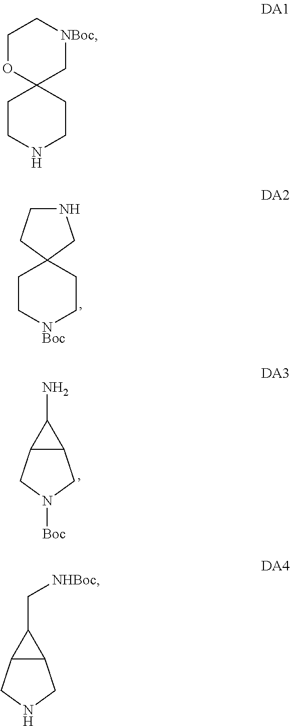

diamine DA1, Stocks et al, Bioorganic and Medicinal Chemistry Letters 2010, p. 7458; diamine DA2, Fritch et al, Bioorganic and Medicinal Chemistry Letters 2010, p. 6375; diamine DA3 and DA4, Moffat et al, J. Med. Chem. 2010, 53, p. 8663-8678). To those familiar in the art, detailed procedures to prepare a variety of mono-protected diamines can also be found in the following references: WO 2004092172, WO 2004092171, and WO 2004092173.

To those skilled in the art, the synthetic sequences shown above in Schemes 2-11 can be repeated with the mono-silylated compound B (shown in Scheme 1) to afford compounds of the Formula II. Also, to those skilled in the art, the synthetic sequences shown in Schemes 2-10 can be repeated on raloxifene to obtain compounds of the Formula III.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Effects of Compounds of the Invention on Nfκb Levels in Raw 264.7 Macrophages

RAW 264.7 cells stably expressing a 3× NFkB response elemement-drive luciferase reporter were seeded into 96 well plates in sera-free medium (Optimem) 18 hours prior to compound application. Compounds of the invention were prepared by first making 100 mM stock solutions in EtOH. Stock solutions were then diluted 1:100 in low LPS FBS (Gemini BenchMark 100-106), mixed vigorously and allowed to incubate at room temperature for 30 minutes. 1:2 serial dilutions were then made in FBS supplemented with 1% EtOH, mixed vigorously, and again allowed to incubate at room temperature for 30 minutes before adding to RAW 264.7 reporter cells (final concentrations: 10% FBS, 100 uM highest compound dilution, 0.1% EtOH) for a 2 hour pretreatment prior to stimulation with LPS. Cells were then stimulated with 200 ng/ml LPS or vehicle control for 3 hours in the presence of the compounds of the invention. A set of six vehicles was left unstimulated with LPS in order to measure the assay floor. AlamarBlue viability dye (Invitrogen) was added to cells simultaneously with the delivery of LPS (final AlamarBlue concentration of 10%). After the 3 h incubation period with LPS, cell viability was measured by reading fluorescence (excitation 550 nm, emission 595 nm) with a Perkin Elmer Victor V plate reader. Then cell media was aspirated from each well. Luciferase signal was then developed by addition of the Britelite Plus reagent (Perkin Elmer). Luciferase activity was measured with the Perkin Elmer Victor V plate reader. NF-κB activity was expressed as a percent of the vehicle control wells (stimulated with LPS). Compounds were tested at 6 dose point titrations in triplicate to determine IC₅₀ values.

As illustrative examples, the IC₅₀ for compounds II-4 and 111-4 were determined to be about 50

### Example 2

### 35Day Ovariectomized (OVX) Rat Assay

Virgin, virus-antibody-free, OVX Sprague-Dawley rats (75 days old) can be purchased from Charles River Laboratories and group housed on a 12 h light:12 h dark cycle with room temperature set at 22° C. The animals all have ad libitum access to both food and tap water. Six animals can be used in each treatment group. Animals are dosed with either the vehicle or with the fatty acid raloxifene derivatives daily for 35 days, beginning on day 4 following ovariectomy. Animals are euthanized by carbon dioxide asphyxiation. The uteri are removed and dissected free of extraneous tissue, and the fluid contents are expelled before determination of wet weight in order to confirm estrogen deficiency associated with ovariectomy. Uterine weight is usually reduced by 75% in response to ovariectomy.

Detailed protocols as well as methods of tissue collection and data analysis can be found in Sato, M. et al. J. Pharm. Exp. Ther. 1995, 272, 252-1259. Briefly, the right femurs are excised and digitized X-rays generated and analyzed at the distal metaphysis. The proximal aspect of the tibia from these animals can be scanned by quantitative computed tomography. Percent protection can be calculated by the following formula: % protection=[(BMD_{test compound}−BMD_{ovx control})/(BMD_{sham control}−BMD_{ovx control})]×100. Statistical evaluations can be made by one-way analysis of variance (ANOVA) and significance is ascribed at a p≦0.05.

### Compounds

The following non-limiting compound examples serve to illustrate further embodiments of the fatty acid raloxifene derivatives. It is to be understood that any embodiments listed in the Examples section are embodiments of the fatty acid raloxifene derivatives and, as such, are suitable for use in the methods and compositions described above.

### Example 3

### Preparation of (S)-4-(6-hydroxy-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)propanoate (II-1)

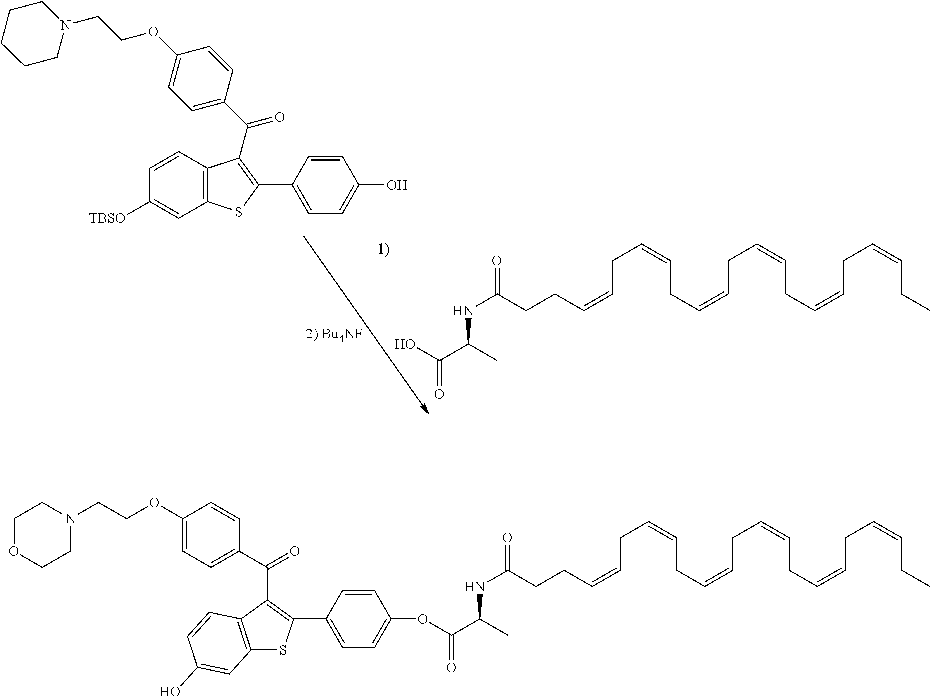

(6-(tert-Butyldimethylsilyloxy)-2-(4-hydroxyphenyl)benzo[b]thiophen-3-yl)(4-(2-(piperidin-1-yl)ethoxy)phenyl)methanone was prepared according to the procedures outlined in Grese et al. J. Med. Chem. 1997, 40, 146-167.

(4Z,7Z,10Z,13Z,16Z,19Z)-docosa-4,7,10,13,16,19-hexaenoic acid (2 mmol) was taken up in CH₃CN (10 mL) along with L-alanine methyl ester (2 mmol) and EDCI (2.2 mmol). The resulting reaction mixture was stirred at room temperature for 2 h and diluted with EtOAc. The organic layer was washed with dilute aqueous NaHCO₃, brine, dried over Na₂SO₄, filtered and concentrated under reduced pressure. Purification by silica gel chromatography (CH₂Cl₂) afforded (S)-methyl 2-((4Z,7Z,10Z,13Z,16Z,19Z)-docosa-4,7,10,13,16,19-hexaenamido)propanoate. This material, (S)-methyl 2-((4Z,7Z,10Z,13Z,16Z,19Z)-docosa-4,7,10,13,16,19-hexaenamido)propanoate was taken up in THF (5 mL) along with 5M aqueous NaOH (3 mL) and the resulting reaction mixture was stirred at room temperature for 2 h. The mixture was concentrated under reduced pressure and the resulting residue was diluted with water and the pH was adjusted to 2 with 5M aqueous HCl. The resulting mixture was extracted with CH₂Cl₂ and the combined organic extracts were washed with brine, dried over Na₂SO₄, filtered and concentrated under reduced pressure to afford (S)-2-((4Z,7Z,10Z,13Z,16Z,19Z)-docosa-4,7,10,13,16,19-hexaenamido)propanoic acid.

In a typical run, (6-(tert-butyldimethylsilyloxy)-2-(4-hydroxyphenyl)benzo[b]thiophen-3-yl)(4-(2-(piperidin-1-yl)ethoxy)phenyl)methanone (0.57 g, 1.42 mmol) was taken up in 15 mL of CH₂Cl₂ along with EDCI (0.34 g, 1.78 mmol), DMAP (0.21 g, 1.78 mmol) and (S)-2-((4Z,7Z,10Z,13Z,16Z,19Z)-docosa-4,7,10,13,16,19-hexaenamido)propanoic acid (0.7 g, 1.18 mmol) under N₂ flow. The resulting reaction mixture was stirred at room temperature for 18 h. It was then diluted with CH₂Cl₂ (20 mL) and washed with brine (3×50 mL). The organic layer was dried over anhydrous Na₂SO₄ and concentrated under reduced pressure. The resulting residue was purified by silica gel chromatography (5% CH₃OH in CH₂Cl₂,) to afford 0.6 g of (S)-4-(6-(tert-butyldimethylsilyloxy)-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)propanoate (Yield: 52%). This material, (S)-4-(6-(tert-butyldimethylsilyloxy)-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)propanoate (0.6 g, 0.618 mmol) was taken up in 5 mL of THF along with tetra-n-butylammonium fluoride (0.161 g, 0.618 mmol). The resulting reaction mixture was stirred at room temperature for 5 minutes. The reaction mixture was concentrated under reduced pressure and purified by silica gel chromatography (10% CH₃OH in CH₂Cl₂,) to afford 180 mg of (S)-4-(6-hydroxy-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)propanoate (Yield: 34%). MS calculated for C₅₃H₆₂N₂O₆S: 855.13. found: 855.0 [M⁺+1].

¹H NMR (400 MHz, CDCl₃) δ 1.15-0.08 (m, 3H), 1.42-1.35 (m, 4H), 1.84-1.47 (m, 5H), 2.10-1.93 (m, 7H), 2.43-2.19 (m, 5H), 2.77-2.74 (m, 10H), 3.18-3.13 (s, 2H), 4.39-4.31 (m, 2H), 4.82-4.39 (m, 1H), 5.35-5.23 (m, 12H), 6.67-6.48 (m, 3H), 6.92-6.784 (m, 2H), 7.19-7.08 (m, 1H), 7.53-7.28 (m, 4H).

### Example 4

### Preparation of (S)-4-(6-hydroxy-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)-3-methylbutanoate

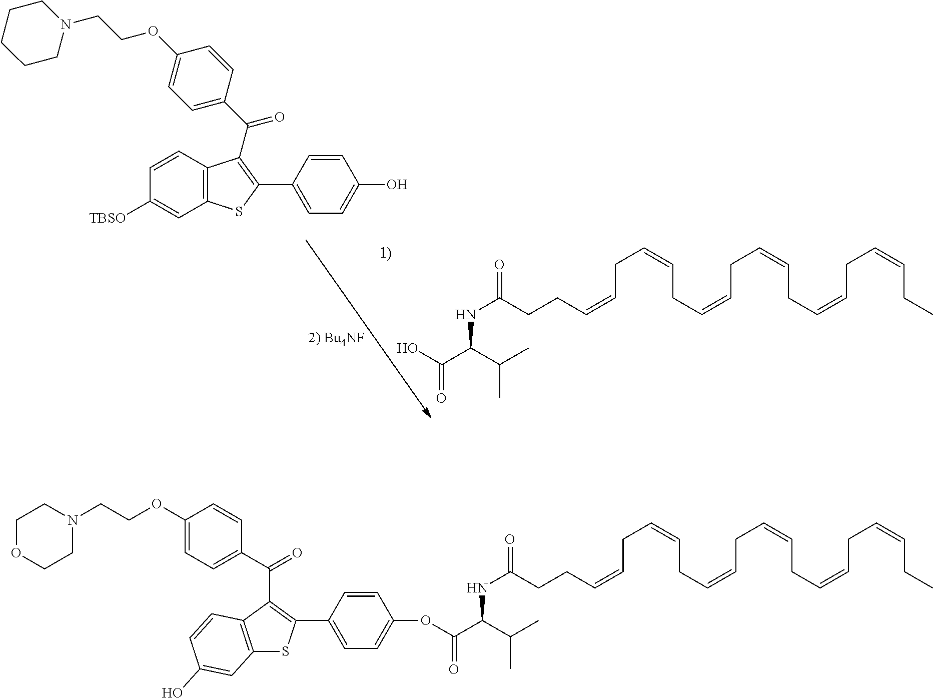

The same experimental procedure outlined above for the synthesis of (S)-4-(6-hydroxy-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)propanoate was used, substituting L-valine methyl ester for L-alanine methyl ester. MS calculated for C₅₅H₆₆N₂O₆S: 883.18. found: 883.5 [M⁺+1].

¹H NMR (400 MHz, CDCl₃) δ 1.15-0.90 (m, 9H), 1.24-1.18 (m, 2H), 1.30 (s, 2H), 1.42-1.38 (m, 4H), 2.03-1.96 (m, 3H), 2.65-2.22 (m, 9H), 2.77-2.74 (m, 12H), 4.08-4.01 (m, 2H), 4.79-4.76 (m, 1H), 5.36-5.20 (m, 12H), 5.95-5.91 (m, 1H), 6.62-6.54 (m, 31H), 7.03-6.870 (m, 1H), 7.31-7.16 (m, 2H), 7.77-7.51 (m, 4H).

### Example 5

### Preparation of 4-(6-hydroxy-3-(4-(2-morpholinoethoxy)benzoyl)benzo[b]thiophen-2-yl)phenyl 3-(4Z,7Z,10Z,13Z,16Z,19Z)-docosa-4,7,10,13,16,19-hexaenamidopropanoate

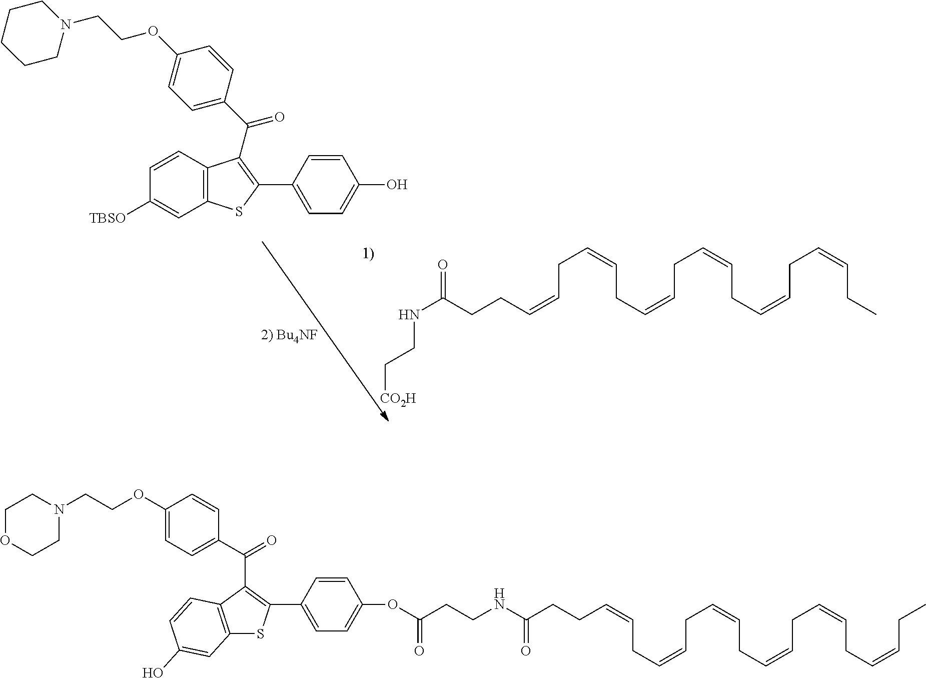

The same experimental procedure outlined above for the synthesis of (S)-4-(6-hydroxy-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)propanoate was used, substituting β-alanine methyl ester for L-alanine methyl ester. MS calculated for C₅₃H₆₂N₂O₆S: 855.13. found: 855.3 [M⁺+1].

¹H NMR (400 MHz, CDCl₃) δ 0.91-0.87 (m, 3H), 1.40-1.37 (s, 2H), 1.60-1.55 (s, 4H), 2.03-1.96 (m, 2H), 2.19-2.15 (m, 2H), 2.48-2.46 (m, 2H), 2.54 (s, 4H), 2.80-2.70 (m, 14H), 3.59-3.55 (m, 2H), 4.05-4.02 (m, 2H), 5.33-5.23 (m, 12H), 6.02 (s, 1H), 6.63-6.54 (m, 4H), 7.01-6.90 (m, 1H), 7.19-7.03 (m, 2H), 7.53-7.52 (m, 1H), 7.74-7.56 (m, 3H).

### Example 6

### Preparation of 4-(6-hydroxy-3-(4-(2-morpholinoethoxy)benzoyl)benzo[b]thiophen-2-yl)phenyl 2-(4Z,7Z,10Z,13Z,16Z,19Z)-docosa-4,7,10,13,16,19-hexaenamidoethylcarbamate

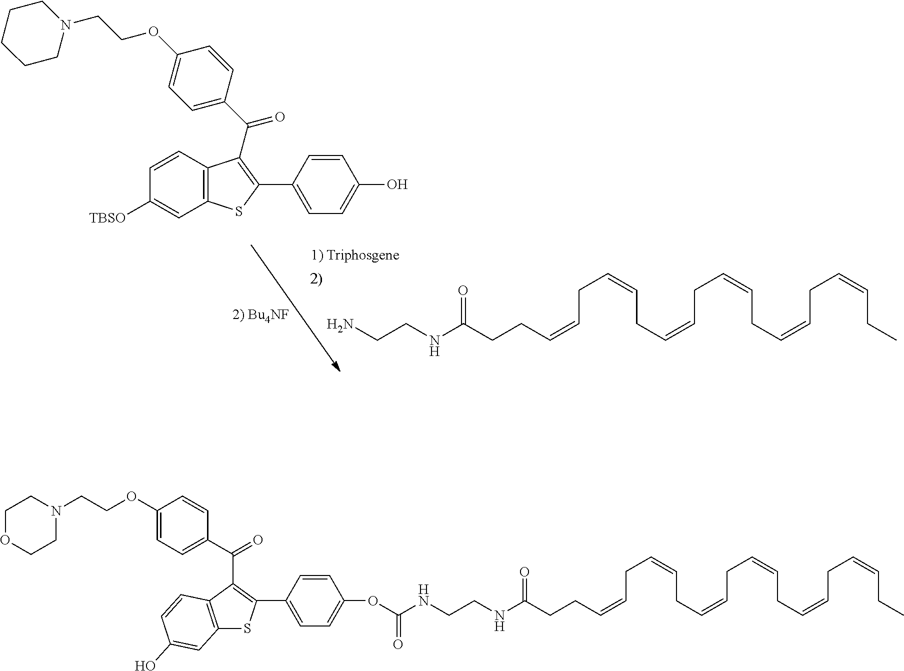

(6-(tert-Butyldimethylsilyloxy)-2-(4-hydroxyphenyl)benzo[b]thiophen-3-yl)(4-(2-(piperidin-1-yl)ethoxy)phenyl)methanone (0.7 g, 1.18 mmol) was taken up in 25 mL of CH₂Cl₂ along with DIEA (0.31 g, 2.37 mmol). 4-Nitrophenyl chloroformate (0.36 g, 1.78 mmol) was then added at room temperature. The resulting reaction mixture was stirred at room temperature for 18 h and diluted with CH₂Cl₂ (20 mL). The organic layer was washed with brine (3×50 mL), dried over anhydrous Na₂SO₄ and concentrated under reduced pressure. The resulting residue was purified by chromatography (5% CH₃OH in CH₂Cl₂) to afford 0.4 g of the nitrophenyl derivative. (Yield: 45%). This material (0.4 g, 0.53 mmol) was taken up in 15 mL of CH₂Cl₂ along with DIEA (0.20 g, 1.59 mmol). The HCl salt of (4Z,7Z,10Z,13Z,16Z,19Z)-N-(2-aminoethyl)docosa-4,7,10,13,16,19-hexaenamide (0.196 g, 0.53 mmol) was then added and the resulting reaction mixture was stirred at room temperature for 18 h. It was then diluted with CH₂Cl₂ (20 mL) and washed with brine (3×50 mL). The organic layer was dried over anhydrous Na₂SO₄ and concentrated under reduced pressure. The resulting residue was purified by chromatography (5% CH₃OH in CH₂Cl₂) to afford 0.25 g of the silylated carbamate derivative. (Yield: 53%). The HCl salt of (4Z,7Z,10Z,13Z,16Z,19Z)-N-(2-aminoethyl)docosa-4,7,10,13,16,19-hexaenamide, in turn, was prepared as follows: (4Z,7Z,10Z,13Z,16Z,19Z)-docosa-4,7,10,13,16,19-hexaenoic acid (1 mmol) was taken up in CH₃CN (5 mL) along with tert-butyl 2-aminoethylcarbamate (1 mmol) and EDCI (1.1 mmol). The resulting reaction mixture was stirred at room temperature for 2 h. It was then washed with brine, dried over Na₂SO₄, filtered and concentrated under reduced pressure. Purification by silica gel chromatography (CH₂Cl₂) afforded tert-butyl 2-(4Z,7Z,10Z,13Z,16Z,19Z)-docosa-4,7,10,13,16,19-hexaenamidoethylcarbamate. tert-Butyl 2-(4Z,7Z,10Z,13Z,16Z,19Z)-docosa-4,7,10,13,16,19-hexaenamidoethylcarbamate (500 mg, 1.06 mmol) was taken up in 4M HCl in dioxane (3 mL). The resulting reaction mixture was allowed to stir at room temperature for 10 min. It was then diluted with EtOAc (10 mL) and concentrated under reduced pressure to afford the HCl salt of (4Z,7Z,10Z,13Z,16Z,19Z)-N-(2-aminoethyl)docosa-4,7,10,13,16,19-hexaenamide.

The silylated carbamate derivative above (0.25 g, 0.25 mmol) was taken up in 3 mL of THF along with tetra-n-butyl ammonium fluoride (TBAF, 0.066 g, 0.25 mmol). The resulting reaction mixture was stirred at room temperature for 5 minutes. The reaction mixture was concentrated under reduced pressure and the resulting residue was purified by silica gel chromatography (10% CH₃OH in CH₂Cl₂,) to afford 180 mg of 4-(6-hydroxy-3-(4-(2-morpholinoethoxy)benzoyl)benzo[b]thiophen-2-yl)phenyl 2-(4Z,7Z,10Z,13Z,16Z,19Z)-docosa-4,7,10,13,16,19-hexaenamidoethylcarbamate (Yield: 35%).

MS calculated for C₅₃H₆₃N₃O₆S: 870.14. found: 870.2 [M⁺+1].

¹H NMR (400 MHz, CDCl₃) δ 0.94-0.81 (m, 9H), 1.5-1.23 (m, 5H), 1.53-1.44 (m, 8H), 1.82-1.78 (m, 6H), 2.2-1.96 (m, 3H), 2.32-2.20 (m, 2H), 2.35-2.34 (m, 2H), 2.77-2.68 (m, 10H), 2.95-2.74 (m, 2H), 3.19-3.15 (m, 4H), 3.36-3.309 (m, 4H), 4.15 (s, 2H), 5.30-5.22 (m, 12H), 6.59-6.56 (m, 3H), 6.89-6.81 (m, 3H), 7.28 (s, 1H), 7.46-7.45 (m, 2H), 7.57-7.55 (m, 2H).

### Example 7

### Preparation of (S)-2-(4-hydroxyphenyl)-3-(4-(2-morpholinoethoxy)benzoyl)benzo[b]thiophen-6-yl 2-((4Z,7Z,10Z,13Z,16Z,19Z)-docosa-4,7,10,13,16,19-hexaenamido)propanoate (III-1)

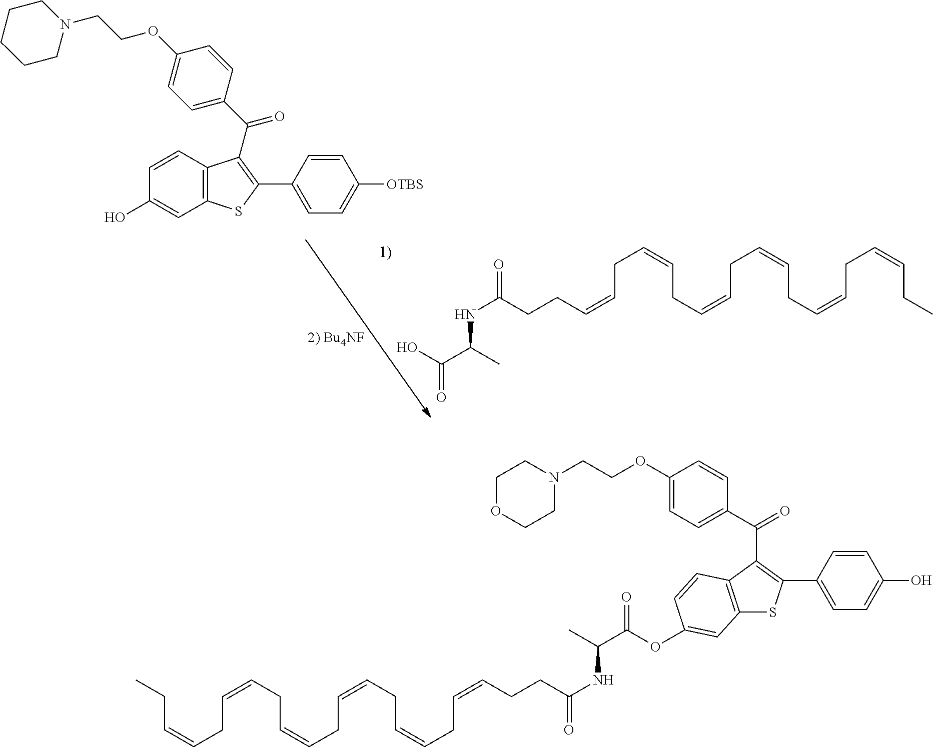

(2-(4-(tert-butyldimethylsilyloxy)phenyl)-6-hydroxybenzo[b]thiophen-3-yl)(4-(2-(piperidin-1-yl)ethoxy)phenyl)methanone was subjected to the same reaction conditions outlined earlier in the synthesis of (S)-4-(6-hydroxy-3-(4-(2-morpholinoethoxy)benzoyl)benzo[b]thiophen-2-yl)phenyl 2-((4Z,7Z,10Z,13Z,16Z,19Z)-docosa-4,7,10,13,16,19-hexaenamido)propanoate. MS calculated for C₅₃H₆₂N₂O₆S: 855.13. found: 855.0 [M⁺+1].

### Example 8

### Preparation of 4-(6-[(2S)-2-((4Z,7Z,10Z,13Z,16Z,19Z)docosa-4,7,10,13,16,19-hexaenoylamino)propanoyloxy]-3-{[4-(2-piperidylethoxy)phenyl]carbonyl}benzo[b]thiophen-2-yl)phenyl (2S)-2-((4Z,7Z,10Z,13Z,16Z,19Z)docosa-4,7,10,13,16,19-hexaenoylamino)propanoate (I-1)

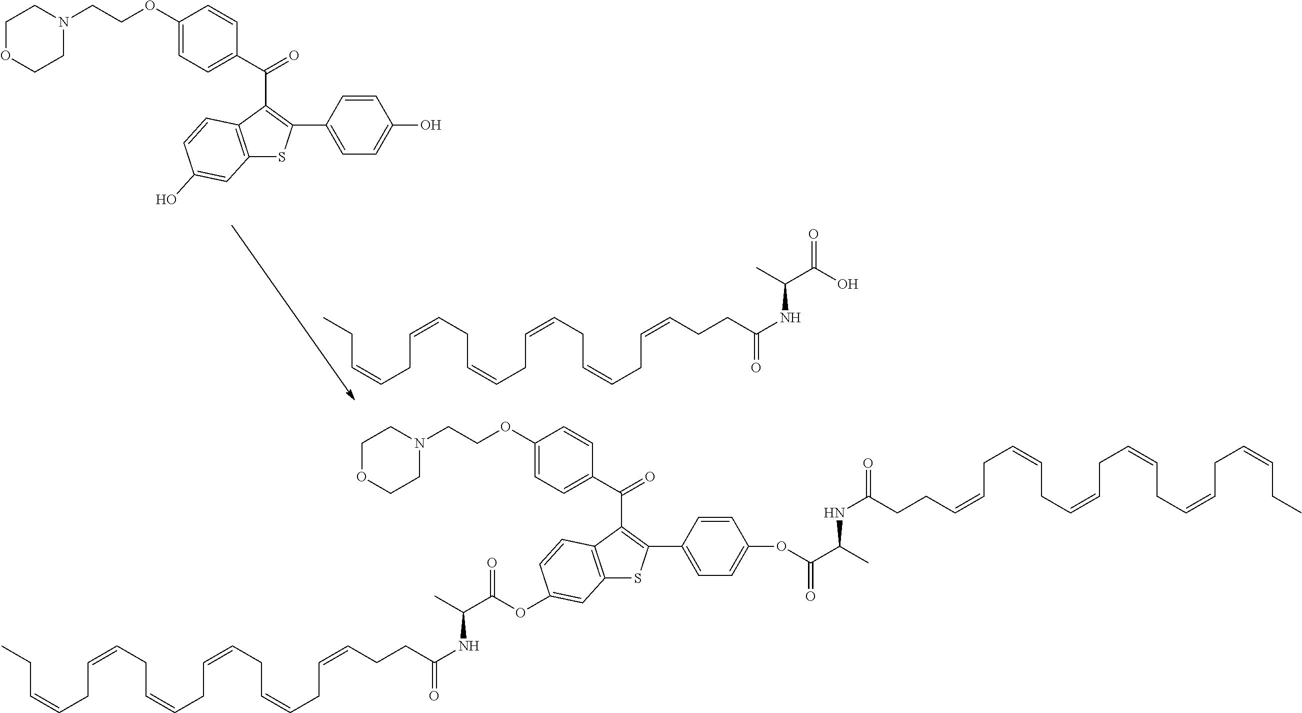

Raloxifene (200 mg, 0.393 mmol) was taken up in 5 mL of DMF along with (S)-2-((4Z,7Z,10Z,13Z,16Z,19Z)-docosa-4,7,10,13,16,19-hexaenamido)propanoic acid (344 mg, 0.862 mmol), HATU (343 mg, 0.90 mmol) and DIEA (240 μL). The resulting reaction mixture was stirred at room temperature for 6 h. It was then diluted with EtOAc (40 mL) and washed with water (4×10 mL), brine, dried (Na₂SO₄) and concentrated under reduced pressure. Purification by chromatography (95% CH₂Cl₂, 5% MeOH) afforded 250 mg of 4-(6-[(2S)-2-((4Z,7Z,10Z,13Z,16Z,19Z)docosa-4,7,10,13,16,19-hexaenoylamino)propanoyloxy]-3-{[4-(2-piperidylethoxy)phenyl]carbonyl}benzo[b]thiophen-2-yl)phenyl (2S)-2-((4Z,7Z,10Z,13Z,16Z,19Z)docosa-4,7,10,13,16,19-hexaenoylamino)propanoate. MS calculated for C₇₇H₉₅N₃O₉S: 1238. found: 1239.0 [M⁺+1].

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A molecular conjugate comprising a raloxifene and a fatty acid covalently linked wherein the fatty acid is selected from omega-3 fatty acids, fatty acids metabolized in vivo into omega-3 fatty acids or lipoic acid, and the conjugate is capable of hydrolysis to produce free raloxifene and free fatty acid with the proviso that the conjugate is not (9Z,12Z,15Z)-4-(6-((9Z,12Z,15Z)-octadeca-9,12,15-trienoyloxy)-3-(4-(2-(piperidin-1-yl)ethoxy)benzoyl)benzo[b]thiophen-2-yl)phenyl octadeca-9,12,15-trienoate.

2. A compound of Formula I: 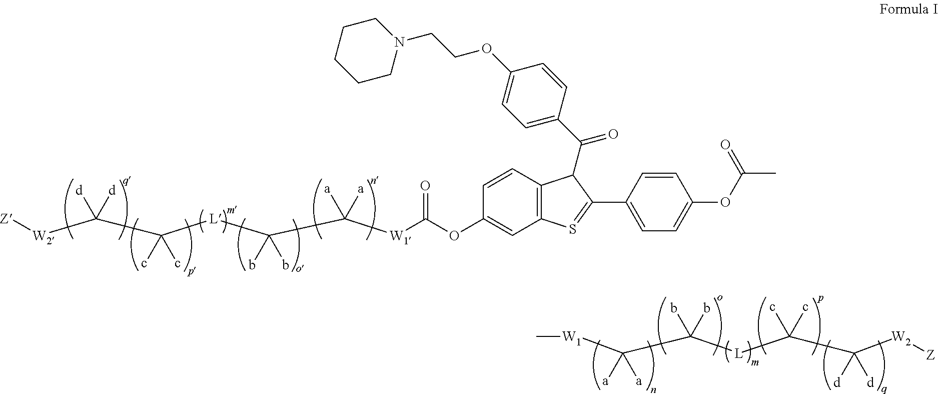
or a pharmaceutically acceptable salt, hydrate, solvate, prodrug, enantiomer or stereoisomer thereof;
wherein
W₁, W_{1′}, W₂, and W_{2′} are each independently null, O, S, NH, NR, or, W₁ and W₂ or W_{1′} and W_{2′} can be taken together can form an imidazolidine or piperazine group,
each a, b, c, and d is independently —H, , —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;
each n, n′, o, o′, p, p′, q, and q′ is independently 0, 1 or 2;
each L and L′ is independently null, —O—, —S—, —S(O)—, —S(O)₂—, —S—S—, —(C₁-C₆alkyl)-, —(C₃-C₆cycloalkyl)-, a heterocycle, a heteroaryl, 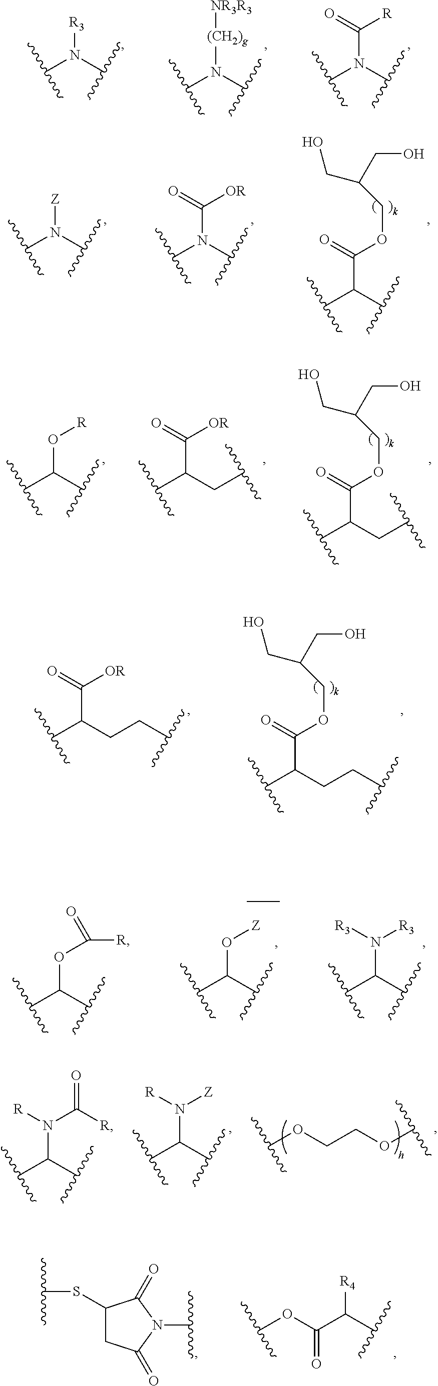 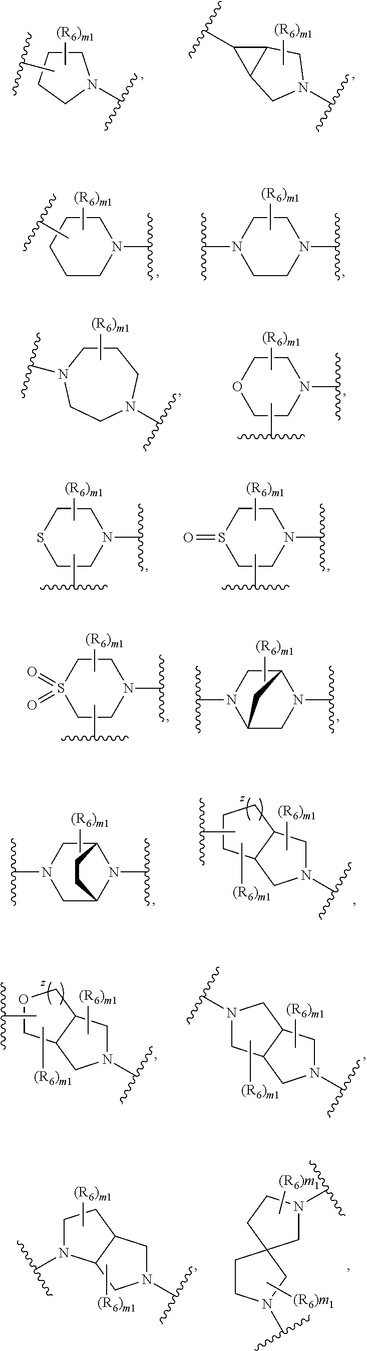 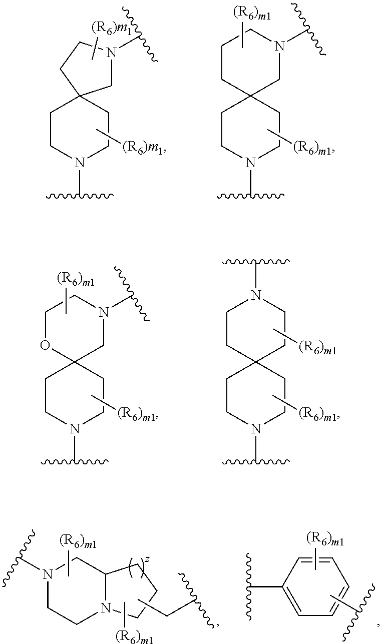
wherein the representation of L is not limited directionally left to right as is depicted, rather either the left side or the right side of L can be bound to the W₁ side of the compound of Formula I;
R₆ is independently —H, , —C₁-C₄ alkyl, gen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2, 3 or 4;
each h is independently 1, 2, 3 or 4;
m and m′ are each independently 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl, or both R₃ groups, when taken together with the nitrogen to which they are attached, can form a heterocycle;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H, 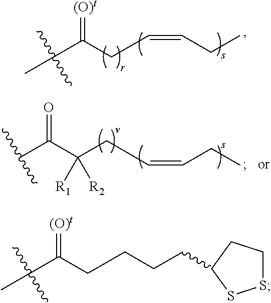
with the proviso that there is at least one 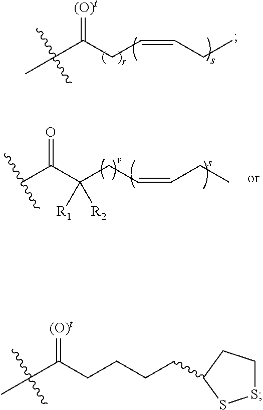
in the compound;
each r is independently 2, 3, or 7;
each s is independently 3, 5, or 6;
each t is independently 0 or 1;
each v is independently 1, 2, or 6;
each R₁ and R₂ are independently hydrogen, deuterium, —C₁-C₄ alkyl, gen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen.
provided that
when m, n, o, p, and q are each 0, W₁ and W₂ are each null, and Z is 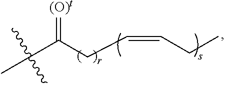
then t must be 0;
when m, n, o, p, and q are each 0, and W₁ and W₂ are each null, then Z must not be 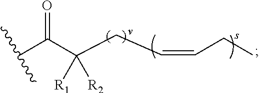
when m′, n′, o′, p′, and q′ are each 0, W_{1′} and W_{2′} are each null, and Z′ is 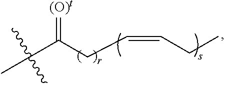
then t must be 0;
when m′, n′, o′, p′, and q′ are each 0, and W_{1′} and W_{2′} are each null, then Z′ must not be 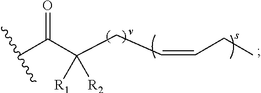
and
when m, m′, n, n′, o, o′, p, p′, q, and q′ are each 0, W₁, W₂, W_{1′}, and W_{2′} are each null, each t is 1, Z and Z′ are each 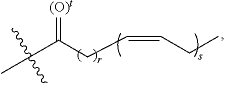
and each r is 7, then one s must be 5 or 6.

3. A compound of Formula II: 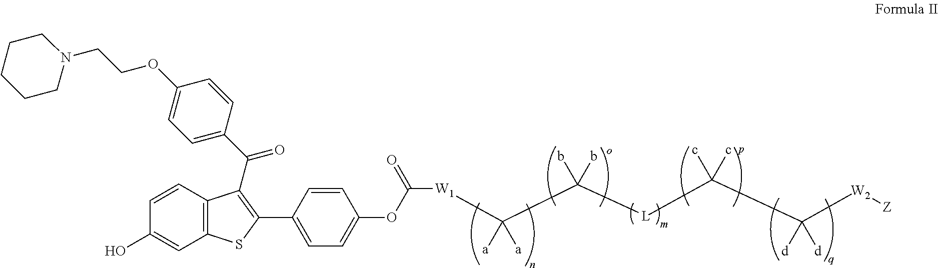
or a pharmaceutically acceptable salt, hydrate, solvate, prodrug, enantiomer or stereoisomer thereof;
wherein
W₁ and W₂ are each independently null, O, S, NH, NR, or W₁ and W₂ can be taken together can form an imidazolidine or piperazine group,
each a, b, c, and d is independently —H, , —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;
each n, o, p, and q is independently 0, 1 or 2;
each L is independently null, —O—, —S—, —S(O)—, —S(O)₂—, —S—S—, —(C₁-C₆alkyl)-, —(C₃-C₆cycloalkyl)-, a heterocycle, a heteroaryl, 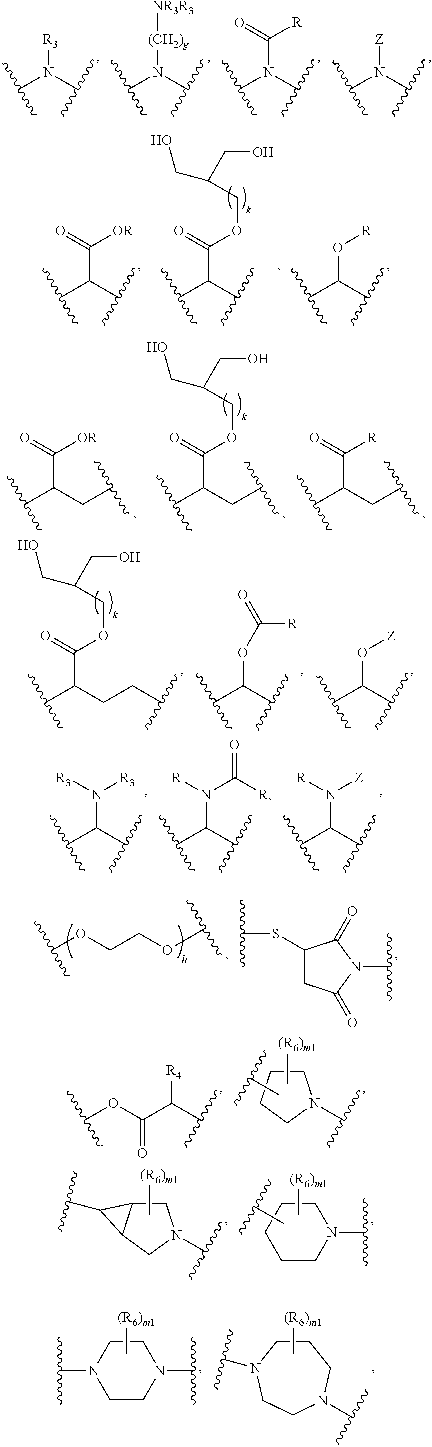 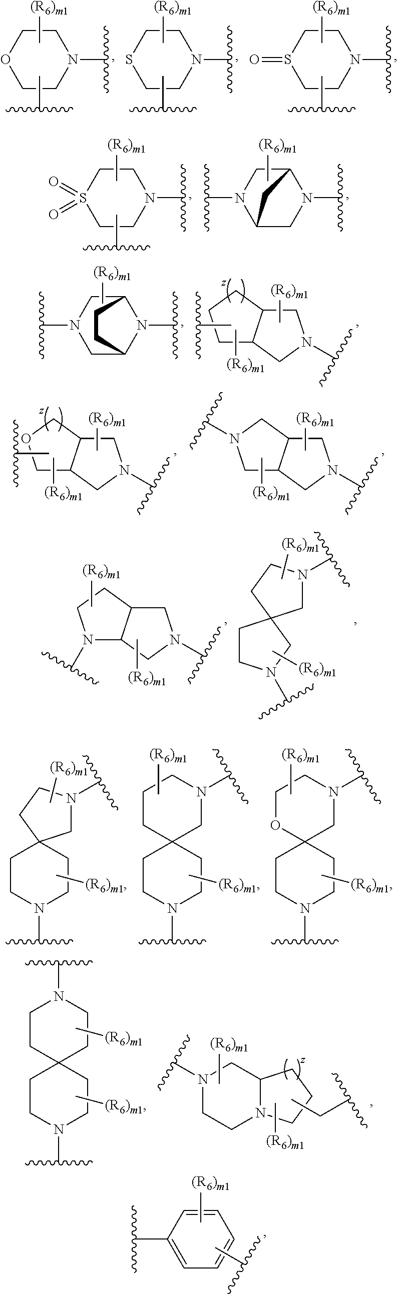
wherein the representation of L is not limited directionally left to right as is depicted, rather either the left side or the right side of L can be bound to the W₁ side of the compound of Formula II;
R₆ is independently —H, , —C₁-C₄ alkyl, gen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2, 3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl, or both R₃ groups, when taken together with the nitrogen to which they are attached, can form a heterocycle;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H, 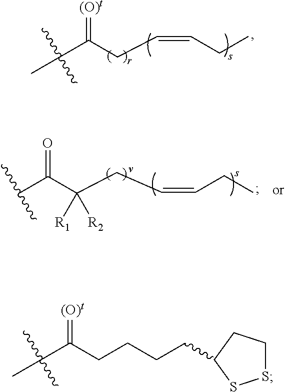
with the proviso that there is at least one 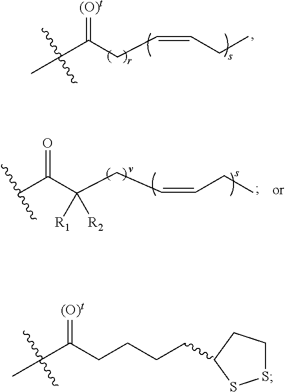
in the compound;
each r is independently 2, 3, or 7;
each s is independently 3, 5, or 6;
each t is independently 0 or 1;
each v is independently 1, 2, or 6;
R₁ and R₂ are each independently hydrogen, deuterium, —C₁-C₄ alkyl, gen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen;
provided that
when m, n, o, p, and q are each 0, W₁ and W₂ are each null, and Z is 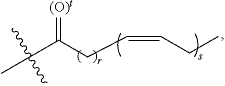
then t must be 0; and
when m, n, o, p, and q are each 0, and W₁ and W₂ are each null, then Z must not be

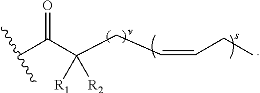

4. A compound of Formula III: 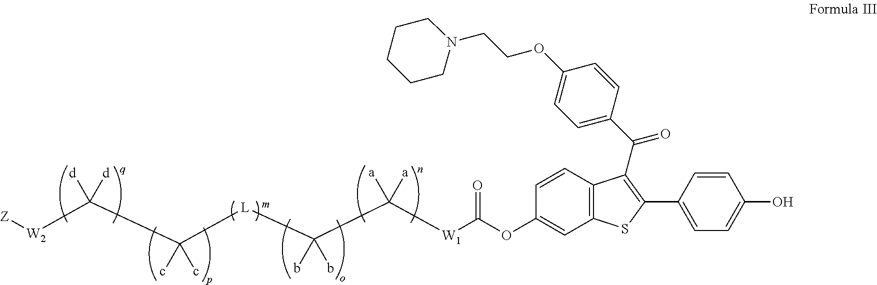
or a pharmaceutically acceptable salt, hydrate, solvate, prodrug, enantiomer or stereoisomer thereof;
wherein
W₁ and W₂ are each independently null, O, S, NH, NR, or W₁ and W₂ can be taken together can form an imidazolidine or piperazine group,
each a, b, c, and d is independently —H, , —CH₃, —OCH₃, —OCH₂CH₃, —C(O)OR, —O—Z, or benzyl, or two of a, b, c, and d can be taken together, along with the single carbon to which they are bound, to form a cycloalkyl or heterocycle;
each n, o, p, and q is independently 0, 1 or 2;
each L is independently null, —O—, —S—, —S(O)—, —S(O)₂—, —S—S—, —(C₁-C₆alkyl)-, —(C₃-C₆cycloalkyl)-, a heterocycle, a heteroaryl, 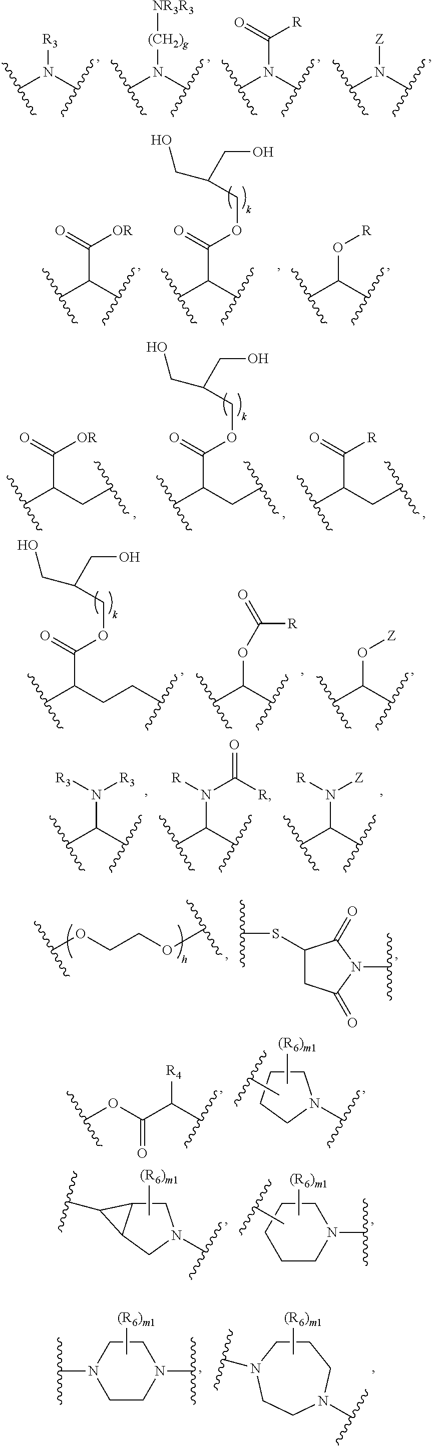 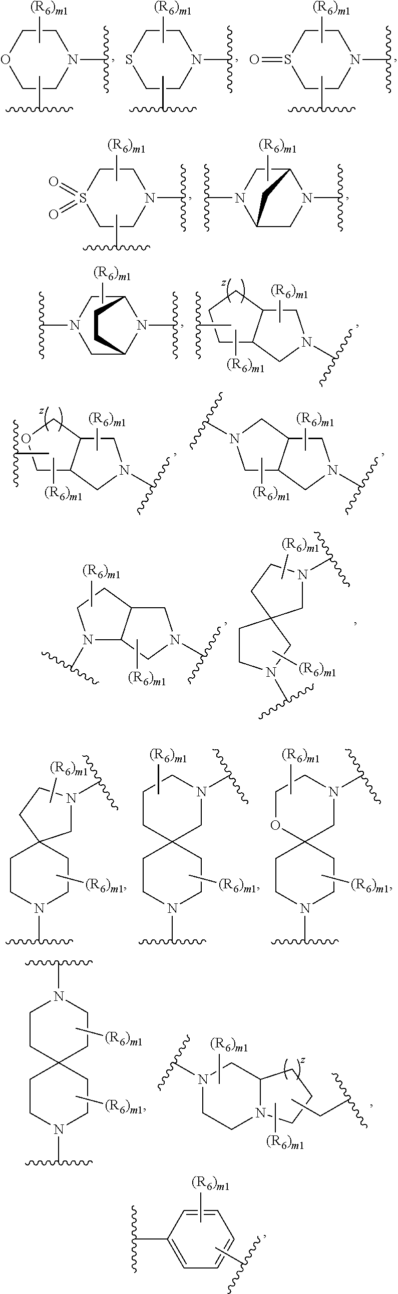
wherein the representation of L is not limited directionally left to right as is depicted, rather either the left side or the right side of L can be bound to the W₁ side of the compound of Formula III;
R₆ is independently —H, , —C₁-C₄ alkyl, gen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2, 3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl, or both R₃ groups, when taken together with the nitrogen to which they are attached, can form a heterocycle;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H; 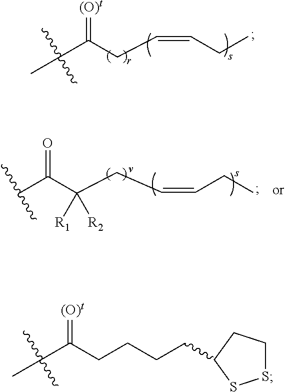
with the proviso that there is at least one 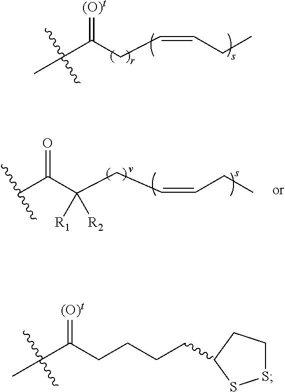
in the compound;
each r is independently 2, 3, or 7;
each s is independently 3, 5, or 6;
each t is independently 0 or 1;
each v is independently 1, 2, or 6;
R₁ and R₂ are each independently hydrogen, deuterium, —C₁-C₄ alkyl, gen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen;
provided that
when m, n, o, p, and q are each 0, W₁ and W₂ are each null, and Z is 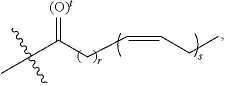
then t must be 0; and
when m, n, o, p, and q are each 0, and W₁ and W₂ are each null, then Z must not be

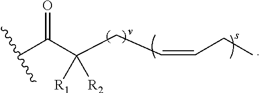

5. A pharmaceutical composition comprising a molecular conjugate of claim 1 and a pharmaceutically acceptable carrier.

6. A pharmaceutical composition comprising a compound of claim 2, 3, or 4 and a pharmaceutically acceptable carrier.

7. A method for treating osteoporosis, endometriosis, uterine fibrosis, metabolic dyslipidemia, or coronary heart disease, the method comprising administering to a patient in need thereof an effective amount of a molecular conjugate of claim 1.

8. A method for lowering the risk of invasive breast cancer in postmenopausal women, the method comprising administering to a patient in need thereof an effective amount of a molecular conjugate of claim 1.

9. A method for treating osteoporosis, endometriosis, uterine fibrosis, metabolic dyslipidemia, or coronary heart disease, the method comprising administering to a patient in need thereof an effective amount of a compound of claim 2.

10. A method for lowering the risk of invasive breast cancer in postmenopausal women, the method comprising administering to a patient in need thereof an effective amount of a compound of claim 2.

11. A method for treating osteoporosis, endometriosis, uterine fibrosis, metabolic dyslipidemia, or coronary heart disease, the method comprising administering to a patient in need thereof an effective amount of a compound of claim 3.

12. A method for lowering the risk of invasive breast cancer in postmenopausal women, the method comprising administering to a patient in need thereof an effective amount of a compound of claim 3.

13. A method for treating osteoporosis, endometriosis, uterine fibrosis, metabolic dyslipidemia, or coronary heart disease, the method comprising administering to a patient in need thereof an effective amount of a compound of claim 4.

14. A method for lowering the risk of invasive breast cancer in postmenopausal women, the method comprising administering to a patient in need thereof an effective amount of a compound of claim 4.
